(19) United States
(12) Patent Application Publication
(10) Pub. No.: US 2004/0058334 A1
(43) Pub. Date: Mar. 25, 2004

(54) Novel method for genetic linkage analysis by mitotic recombination

(76) Inventors: Frederick S. Kaplan, Philadelphia, PA (US); Eileen M. Shore, Washington, PA (US); Scott M. Williams, Nashville, TN (US)

Correspondence Address: NATH & ASSOCIATES, 1030 15th STREET, 6TH FLOOR, WASHINGTON, DC, 20005, US

(21) Appl. No.: 10/252,959

(22) Filed: Sep. 24, 2002

(51) Int. Cl. C12Q 1/68; C12N 5/06; C12N 15/85

(52) U.S. Cl. 435/6; 435/325; 435/455

(57) ABSTRACT

This invention relates to a novel method for genetic linkage analysis and identification of a mutated gene by mitotic recombination. In particular, this invention relates to a method for genetic linkage analysis and identification of a mutated gene linked with the rare autosomal dominant disorder Fibrodysplasia Ossificans Progressiva. This invention also relates to the defective and/or mutated genes for Fibrodysplasia Ossificans Progressiva identified by Applicants' novel methods. Kits and apparatus for genetic linkage analysis and identification of a mutated gene are also provided.

This invention was made with government support under grant 1 R21 AR 48429-01, awarded by the National Institute of Arthritis and Musculoskeletal and Skin Diseases of the National Institutes of Health. Accordingly, the government has certain rights in the invention.

### BACKGROUND OF THE INVENTION

1. Field of Invention

The present invention relates to a novel method for genetic linkage analysis by mitotic recombination. For rare genetic disorders and other uncharacterized disorders, family pedigrees are scarce, and genetic linkage and positional cloning are difficult, expensive, time consuming, inefficient, and/or impossible. Applicants therefore have devised a novel and innovative alternate approach using mitotic recombination in somatic cells. By enhancing the production of mitotically-generated recombinant progeny cells, this method will create a large cellular pseudo-family from a single “parent” cell line. Somatic cell recombination will create a loss of the mutant chromosomal locus in a subset of the cellular “progeny”, which will be identified by the loss of the marker for the mutant phenotype. The chromosomal site of recombination will be identified using chromosomal microsatellite markers within these “progeny” cells, which will facilitate a highly focused and targeted positional cloning approach to identify the mutated gene locus in patients having such disorders.

2. Background

Great advancements in developing therapeutics and cures for inherited disorders will result from identification of the gene mutations that cause these disorders. For common genetic disorders, large pedigrees that show the disease trait inherited from one generation to the next can be produced, then analyzed by genome-wide linkage analyses followed by positional cloning or database searching to identify the mutated gene(s). On the other hand, most of the 3000 or so inherited human disorders that have been described are relatively rare. For such genetic disorders, family pedigrees are scarce, and genetic linkage and positional cloning are difficult, inefficient, and/or impossible. In such rare disorders, the paucity of multi-generational families may also render doubt about the fidelity of localization(s) which that are determined by genetic linkage analysis, as genetic heterogeneity may exist.

In the case of genetic disorders which have not yet been characterized, the simple and efficient methods for genetic linkage analysis of the present invention will identify the gene mutation(s) that cause these disorders without the time-consuming process, and expense, involved in the conventional collection of detailed family pedigrees, genetic linkage analysis, and positional cloning.

3. Related Art

In traditional Mendelian genetic mapping experiments, linkage is analyzed in homologous recombination in meiosis using various (i.e. two-point and three-point) testcrosses. While useful for laboratory specimens, such techniques are virtually useless in human subjects because planned breeding experiments are not possible for obvious ethical reasons.

A representative conventional linkage mapping or linkage analysis approach for determining whether one disease marker is linked to another disease marker, and isolating a mutant gene, consists generally of the following steps:

(a) collection of detailed family pedigrees of affected individuals and examining segregation patterns in each family pedigree;

(b) identifying a marker which segregates with a disease, disorder, or condition of interest;

(c) genetic linkage analysis, by statistical methods, of the recombination frequency of the disease marker with other, known marker(s) to isolate a target chromosomal location; and

(d) positional cloning of the target chromosomal location by (1) cloning DNA sequences from the identified chromosomal location and using such cloned sequences to probe for genes in the chromosomal location, often using analysis of the target chromosomal location by RFLP or VNTR analysis and chromosome walking or chromosome jumping (2) sequencing isolated genes, and (3) comparing the sequence of isolated genes to known “normal” sequences to identify mutation(s). More recently, the availability of genomic databases has reduced the amount of work in this last step.

When the metabolic basis is known for a genetic inherited disease, that information can be used directly to clone the gene involved. In the vast majority of cases in which the metabolic basis is not known, genome wide linkage analysis and positional cloning approaches have been successful in identifying the damaged genes for a number of genetic diseases, for example cystic fibrosis. However, the success of these strategies is greatly dependent on the identification of large multigenerational pedigrees. Such families are needed in order to produce statistically meaningful linkage analysis data and to accurately predict the chromosomal locus containing the mutated gene(s) in the disease, disorder, or condition of interest. Successful genetic linkage is dependent on the ability of researchers to obtain DNA samples from all critical members of these families. Further difficulties in these studies can arise if a disorder exhibits genetic heterogeneity (i.e. is caused by mutations at different genetic loci in different families).

Interspecific somatic cell hybrids have also been developed using human donor cells with mouse, hamster, or rat recipient cells. Such hybrids begin with the fusion of donor and recipient cells to produce heterokaryons; nuclear fusion produces hybrid chromosomes, and subsequent cell proliferation, under selection to eliminate unfused cells, results in the loss of non-hybridized donor chromosomes, ultimately resulting in hybrids having one or a few donor chromosome(s) or chromosome fragment(s). The process of producing hybrids is random, and thus a large number of hybrids must be produced and screened in order to isolate a single gene of interest.

U.S. Pat. No. 5,449,604 describes an exemplary method for isolating a DNA segment indicative of a disease trait in a family population, wherein said family population consists essentially of a plurality of blood relatives of an individual having a known disease trait, comprising the steps of:

(a) preparing a test sample of immobilized separated genomic DNA fragments from a plurality of the blood relatives,

(b) contacting each of the test samples with a test oligonucleotide under conditions permitting hybridization of complementary single stranded DNA molecules, wherein the test oligonucleotide specifically hybridizes to a nucleotide sequence between markers a and b,

(c) identifying a plurality of hybridized molecules so formed as alleles of the genetic marker in the family population,

(d) identifying one of the genetic marker alleles as indicative of the disease trait in the family population by either

(e) determining by pedigree analysis a segregation value for each of the genetic marker alleles and the disease trait, and selecting an indicative genetic marker allele that co-segregates with the disease trait in the family population, or

measuring genetic linkage between each of the genetic marker alleles and the disease trait, and selecting a genetic marker allele as indicative of the disease trait in the family population if the selected genetic marker allele has a maximal LOD score of at least 3 at a recombination fraction of about 0.0 to about 0.1 for genetic linkage with the disease trait in the family population, and

(f) isolating a DNA segment comprising the indicative genetic marker allele.

It is notable that the marker identified in U.S. Pat. No. 5,449,604, D14S43, was selected at random as a marker for chromosome 14 and fortuitously gave highly significant positive LOD scores in the disease of interest, early-onset non-Volga German Alzheimer's Disease.

U.S. Pat. No. 5,449,604 describes an additional exemplary method for the genotypic diagnosis of the presence of a mutated gene in symptomatic or at risk individuals or fetuses belonging to a family suspected of carrying said mutated gene comprising:

a) detecting the presence or absence of DNA polymorphisms genetically linked to the mutated gene, wherein said polymorphisms are located in a known genetic interval of a chromosome, and flanked by and including known polymorphic microsatellite markers; and

b) determining the risk to the individual or fetus of carrying said mutated gene based on the presence or absence of said polymorphisms, thereby providing a genotypic diagnosis.

It is notable that the genetic linkage analysis found in U.S. Pat. No. 5,449,604 began with 2 large CADASIL pedigrees, which initially assigned the CADASIL locus to chromosome 19. Multilocus analysis with the location scores method then established the best estimate for the location of the gene within a 14 cM interval bracketed by D19S221 and D19S215 loci. (See Nature Genetics, 3:256-259, (1993) and Nature Genetics, 5:40-46 (1993)).

Thus, the techniques currently available for linkage analysis and isolation of mutated genes suffer common limitations: high cost and slow processing speed. A genetic linkage approach that completely circumvents the need to obtain DNA samples from many large pedigrees will greatly expand the ability of researchers to identify the genetic causes of diseases, disorders, or conditions for which such families cannot be, or have not been, obtained. Applicants' novel inventive strategy was developed out of necessity, and inspired by the extreme frustration of not identifying enough multigenerational families to perform a definitive linkage analysis for the disorder Fibrodysplasia Ossificans Progressiva (FOP).

### SUMMARY OF THE INVENTION

The present invention relates to a novel method for genetic linkage analysis by mitotic recombination. By enhancing the production of mitotically-generated recombinant progeny cells, this method will create a large cellular pseudo-family from a single “parent” cell line. Somatic cell recombination will create a loss of the mutant chromosomal locus in a subset of the cellular “progeny”, which will be identified by the loss of the marker for the mutant phenotype. The chromosomal site of recombination will be identified using chromosomal microsatellite markers within these “progeny” cells, which will facilitate a highly focused and targeted positional cloning approach to identify the mutated gene locus in patients having such disorders.

Thus, the present invention relates to a method for genetic linkage analysis, comprising:

(a) identifying one or more disease phenotype marker(s), each of which is linked to a disease, disorder, or condition of interest;

(b) inducing mitotic recombination in a parent cell line having a mutated gene to produce recombinant progeny cells;

(c) screening said progeny cells for recombinant cellular DNA by selecting those cells having a loss or gain of one or more of said disease phenotype marker(s); and

(d) identifying chromosomal site(s) of recombination within said progeny cells having a loss or gain of disease phenotype marker(s).

Additionally, the present invention relates to a method for identification of a mutated gene, comprising:

(a) identifying one or more disease phenotype marker(s), each of which is linked to a disease, disorder, or condition of interest;

(b) inducing mitotic recombination in a parent cell line having a mutated gene to produce recombinant progeny cells;

(c) screening said progeny cells for recombinant cellular DNA by selecting those cells having a loss or gain of one or more of said disease phenotype marker(s); and

(d) identifying chromosomal site(s) of recombination within said progeny cells having a loss or gain of disease phenotype marker(s);

(e) identifying one or more candidate gene(s) for said mutated gene within said chromosomal site(s) of recombination; and

(f) screening each said candidate gene for mutations.

In particular, the present invention relates to a method for genetic linkage analysis, comprising:

(a) identifying one or more disease phenotype marker(s), each of which is linked to a disease, disorder, or condition of interest;

(b) inducing mitotic recombination in a parent cell line having a mutated gene to produce recombinant progeny cells,

wherein said mitotic recombination is induced by induced by treatment of a parent cell with 12-0-tetradecanolyphorbol-13-acetate;

(c) screening said progeny cells for recombinant cellular DNA by selecting those cells having a loss or gain of one or more of said disease phenotype marker(s),

wherein said loss of one or more disease phenotype marker(s) is produced by a loss of heterozygosity within said progeny cells; and

(d) identifying chromosomal site(s) of recombination within said progeny cells having a loss or gain of disease phenotype marker(s),

wherein said chromosomal site(s) of recombination within said progeny cells having a loss of disease phenotype marker(s) are identified using chromosomal microsatellite markers.

The present invention further relates to a method for genetic linkage analysis and identification of a mutated gene linked with Fibrodysplasia Ossificans Progressiva, comprising:

(a) inducing mitotic recombination in a parent cell line having a mutated gene linked with Fibrodysplasia Ossificans Progressiva to produce recombinant progeny cells,

wherein said mitotic recombination is induced by induced by treatment of a parent cell with 12-0-tetradecanolyphorbol-13-acetate;

(b) screening said progeny cells for recombinant cellular DNA by selecting those cells having a loss of BMP4 over-expression, loss of BMP antagonist response to a BMP4 stimulus, or decrease in the density of BMP receptor BMPRIA,

wherein said loss of BMP4 over-expression, loss of BMP antagonist response to a BMP4 stimulus, or decrease in the density of BMP receptor BMPRIA is produced by a loss of heterozygosity within said progeny cells;

(c) identifying chromosomal site(s) of recombination within said progeny cells having a loss of BMP4 over-expression, loss of BMP antagonist response to a BMP4 stimulus, or decrease in the density of BMP receptor BMPRIA,

wherein said chromosomal site(s) of recombination within said progeny cells having a loss of BMP4 over-expression, loss of BMP antagonist response to a BMP4 stimulus, or decrease in the density of BMP receptor BMPRIA are identified using chromosomal microsatellite markers;

(d) identifying one or more candidate gene(s) for said mutated gene linked with Fibrodysplasia Ossificans Progressiva within said chromosomal site(s) of recombination within said progeny cells,

wherein each said candidate gene is identified by positional cloning or database searching; and

(e) screening each said candidate gene for mutations.

The present invention further relates to a kit for producing a clonally-expanded, nested cellular progeny cell set from a parent cell, comprising:

(a) a means for obtaining and isolating a parent cell line from an individual;

(b) a means for inducing mitotic recombination in said parent cell line to produce progeny cells;

(c) a means for growing said progeny cells; and

(d) a means for screening said progeny cells for recombinant progeny cells.

The present invention further relates to an apparatus for genetic linkage analysis of a cell of an organism having a genetic mutation of interest and a linked disease phenotype marker, comprising:

(a) a first stage comprising a system for inducing mitotic recombination in a parent cell line to produce clonally-expanded progeny cells;

(b) a second stage comprising a cell sorting system for selection for the loss or gain of expression of said disease phenotype marker;

(c) a third stage comprising an antibiotic selection system for selection of cells that contain said disease phenotype marker;

(d) a fourth stage comprising an isolation system for isolating genomic DNA from said progeny cells;

(e) a fifth stage comprising a screening system for screening said genomic DNA for one or more genomic marker(s); and

(f) a sixth stage comprising a system for correlating said sites of homologous recombination and loss or gain of said mutant phenotype with the genomic DNA sequence of said organism.

The present invention further relates to a DNA sequence containing a mutation, identified by a process comprising:

(a) inducing mitotic recombination in a parent cell line having a mutated gene linked with Fibrodysplasia Ossificans Progressiva to produce recombinant progeny cells,

wherein said mitotic recombination is induced by induced by treatment of a parent cell with 12-0-tetradecanolyphorbol-13-acetate;

(b) screening said progeny cells for recombinant cellular DNA by selecting those cells having a loss of BMP4 over-expression, loss of BMP antagonist response to a BMP4 stimulus, or decrease in the density of BMP receptor BMPRIA, p2 wherein said loss of BMP4 over-expression, loss of BMP antagonist response to a BMP4 stimulus, or decrease in the density of BMP receptor BMPRIA is produced by a loss of heterozygosity within said progeny cells;

(c) identifying chromosomal site(s) of recombination within said progeny cells having a loss of BMP4 over-expression, loss of BMP antagonist response to a BMP4 stimulus, or decrease in the density of BMP receptor BMPRIA,

wherein said chromosomal site(s) of recombination within said progeny cells having a loss of BMP4 over-expression, loss of BMP antagonist response to a BMP4 stimulus, or decrease in the density of BMP receptor BMPRIA are identified using chromosomal microsatellite markers;

(d) identifying one or more candidate gene(s) for said mutated gene linked with Fibrodysplasia Ossificans Progressiva within said chromosomal site(s) of recombination within said progeny cells,

wherein each said candidate gene is identified by positional cloning or database searching;

(e) sequencing each said candidate gene;

(f) comparing the DNA sequence for each candidate gene with a wild-type DNA sequence of said gene; and

(g) identifying said DNA sequence containing a mutation for each said candidate gene.

The present invention further relates to a method for treating Fibrodysplasia Ossificans Progressiva, comprising:

(a) identifying a defective gene responsible for causing Fibrodysplasia Ossificans Progressiva;

(b) harvesting somatic parent cells from an individual having a mutated gene producing the phenotype for Fibrodysplasia Ossificans Progressiva;

(c) producing recombinant progeny cells by a method of inducing mitotic recombination in said parent cells line having a mutated gene;

(d) screening said recombinant progeny cells for recombinant cells having a wild-type copy of said defective gene; and

(e) re-introducing said recombinant cells having a wild-type copy of said defective gene into said individual.

Finally, the present invention relates to a method for treating Fibrodysplasia Ossificans Progressiva, comprising:

(a) identifying a defective gene responsible for causing Fibrodysplasia Ossificans Progressiva; and

(b) administering a composition which upregulates the expression of a wild-type gene, down regulates the expression of said defective gene, or indirectly sequesters or deactivates a defective protein produced by expression of said defective gene.

### BRIEF DESCRIPTION OF THE DRAWINGS

FIG. 1 is a drawing which depicts an exemplary process for the generation of a cellular pseudo-family, from a small FOP kindred.

FIG. 2 is a drawing which depicts an exemplary process for mitotic recombination and loss of heterozygosity (LOH).

FIG. 3 is a drawing which depicts an exemplary process for using microsatellite markers to detect sites of homologous recombination.

FIG. 4 is a drawing which depicts the result of an exemplary process for generating a nested set of marker LOH and a phenotype of interest, using FOP as the disorder of interest.

### DETAILED DESCRIPTION OF THE INVENTION

Definitions

“Gene” broadly refers to any segment of DNA which codes for an RNA molecule and/or a polypeptide associated with a biological function. Genes include coding sequences and the regulatory sequences required for their expression. Genes also include nonexpressed DNA segments that, for example, form introns or recognition sequences for other proteins.

“Genetic linkage” refers to an association of two or more non-allelic genes which do not show independent assortment, often due to physical association on the same chromosome.

“Linkage analysis” refers to a method which is understood by those skilled in the art for determining whether one marker is linked to another marker. Several common methods exist for determining whether one marker is linked to another marker. “Linkage analysis” conventionally refers to a method of genotyping polymorphic markers in human families by examining segregation patterns in a family pedigree, then analyzing by statistical methods, such as by an LOD score, the recombination of one marker with a known marker already on a genetic map.

“Genotype analysis” refers to a method for determining the presence or absence of one or more allelic variants of a genetic marker in the genomic DNA prepared from an individual. In a population, there will generally be at least two major alleles for genes in genomic DNA, but for repetitive sequence elements there may be more than two DNA allelic variants of a marker.

“Map unit” or “centi-Morgan” (“cM”) refers to a quantitative measure of the genetic “distance” between two gene pairs on a genetic map, which is determined by a recombinant frequency of one percent (1%). These terms describe a statistical relationship between genes, not a physical distance which is measured in, for example, nucleic acid base pairs, as might be determined by DNA sequencing.

“Screening” refers to a process in which (1) it is determined which cells do and do not express a screening marker or phenotype (or a selected level of marker or phenotype), and (2) physically separates the cells having the desired property.

“Selection” is a form of screening in which identification and physical separation are achieved simultaneously by expression of a selection marker, which, for example, allows cells expressing the marker to survive while other cells die, or vice versa. Exemplary screening markers include luciferase, β-galactosidase, and fluorescent proteins such as green fluorescent protein (GFP). Exemplary selection markers include drug and toxin resistance genes.

“Mitotic recombination” refers to a reciprocal exchange of genetic material between non-sister chromatids of homologous chromosomes during mitosis in somatic cells.

“Somatic cell” refers to any cell of an organism other than a germ cell.

“Parent cell” refers to an individual cell, or member of a cell line, which displays one or more characteristic(s) of interest and from which other cells or cell lines are derived. “Progeny cell” refers to a cell or cell line derived from a “parent cell.”

“Cellular family” or “cellular pseudo-family” refers to a group of cells which are closely related, but are biologically engineered to produce structural and/or characteristic variations. Such variations are often intended to mimic those found to occur naturally over many generations in a family pedigree.

“Pedigree” or “family pedigree” refers to a family tree annotated with the inheritance patterns of one or more traits, usually established through compilation and analysis of phenotypic records over several generations.

“Positional cloning” refers to the process for identification of a gene responsible for a genetic disorder, wherein the DNA of a chromosomal site is cloned and compared to the corresponding of normal individual(s), usually by sequence analysis.

“Recombination” refers to the process in which a new genotype is formed by reassortment of genetic material.

“Homologous recombination” refers to the reciprocal exchange of chromosome segments between homologous chromosomes based on sequence homology. The term embraces both crossing over and gene conversion.

“Phenotype” refers to the observable functional and/or structural characteristics of an organism. “Mutant phenotype” refers to the observable functional and/or structural characteristics of an organism having a genetic mutation which is expressed.

“Marker” refers to a nucleotide sequence that is present in genomic DNA and which is identifiable with specific oligonucleotide probes in the DNA of different individuals in a human population. Representative examples of markers include genes, transcription regulatory elements, repetitive sequence motifs, i.e., short and long tandem repeat sequences (SLTR; di-, tri-, and longer repeated sequences), and the like.

“Genetic marker” refers to a phenotypic difference that is used to analyze the genetic characteristics of an organism.

“Selectable marker” refers to a nucleic acid sequence, the expression of which allows cells containing the nucleic acid sequence to be identified on a particular medium. A selectable marker can be one that allows a cell to proliferate on a medium that prevents or slows the growth of cells without the nucleic acid sequence. Examples include antibiotic resistance genes and genes which allow an organism to grow on a selected metabolite. Alternatively, the nucleic acid sequence can facilitate visual screening of transformants by conferring on cells a phenotype that is easily identified. Such an identifiable phenotype may be, for example, the production of luminescence or the production of a colored compound, or the production of a detectable change in the medium surrounding the cell.

“Microsatellite” refers to a repeating DNA sequence having variable number tandem repeats (“VNTR“) units, generally of two to four base pairs. “Microsatellite marker” refers to a repeating DNA sequence that is used to analyze the genetic characteristics of an organism.

“Mutation” refers to the process in which genetic material undergoes a detectable and heritable structural change. “Mutated gene” refers to a gene in which the structure of the gene is altered at the molecular level.

“RFLP” refers to restriction fragment length polymorphism, a variation in the size of the DNA fragments produced by the action of restriction endonuclease.

“VNTR” refers to variable number tandem repeats, a multi-allelic DNA polymorphism that results from insertions or deletions of DNA between restriction sites.

“PCR” refers to the polymerase chain reaction process for the amplification of DNA. The process amplifies DNA segments through the use of a DNA template molecule, 2 oligonucleotide primers, and a DNA polymerase enzyme, which is repeated 30-40 times to amplify and enrich the template-specific molecules in the reaction product.

“Homologous chromosomes” refers to a distinct pair of chromosomes which are generally identical with respect to the arrangement of genes which they contain, which appear visually identical, and which pair during meiosis.

“Autosomal” refers to a characteristic of a non-sex chromosome. “Autosomal dominant disorder” refers to a genetic disorder which produces the same characteristic or phenotype whether the mutant gene is present in the heterozygous state or the homozygous state.

“Wild-type” refers to the phenotype which is naturally exhibited by the majority of members of a population. It is to be understood that some variation in a wild-type nucleic acid sequence is to be expected, and the salient feature is normal protein function.

“Loss of heterozygosity” refers to the process in which the single allele in a heterozygous dominant mutant is modified in a manner which results in the loss of the dominant phenotype.

“Polymorphism” refers to a subject marker which can be found in distinguishably different physical forms (e.g., size, charge, nucleotide sequence) in genomic DNA obtained from different individuals in a human population.

“Polymorphic” refers to the regular and simultaneous existence in a population of two or more genotypes at a particular genetic locus.

“Uniparental isodisomy” refers to the presence of two identical chromosome segments having a single parental origin.

“Effecting” refers to the process of producing an effect on biological activity, function, health, or condition of an organism in which such biological activity, function, health, or condition is maintained, enhanced, diminished, or treated in a manner which is consistent with the general health and well-being of the organism.

“Enhancing” the biological activity, function, health, or condition of an organism refers to the process of augmenting, fortifying, strengthening, or improving.

“Pharmaceutically acceptable salt, ester, or solvate” refers to a salt, ester, or solvate of a subject compound which possesses the desired pharmacological activity and which is neither biologically nor otherwise undesirable. A salt, ester, or solvate can be formed with inorganic acids such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, gluconate, glycerophosphate, hemisulfate, heptanoate, hexanoate, hydrochloride, hydrobromide, hydroiodide, 2-hydroxyethanesulfonate, lactate, maleate, methanesulfonate, naphthylate, 2-naphthalenesulfonate, nicotinate, oxalate, sulfate, thiocyanate, tosylate and undecanoate. Examples of base salts, esters, or solvates include ammonium salts; alkali metal salts, such as sodium and potassium salts; alkaline earth metal salts, such as calcium and magnesium salts; salts with organic bases, such as dicyclohexylamine salts; -methyl-D-glucamine; and salts with amino acids, such as arginine, lysine, and so forth. Also, the basic nitrogen-containing groups can be quarternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; aralkyl halides, such as benzyl and phenethyl bromides; and others. Water or oil-soluble or dispersible products are thereby obtained.

It is to be understood that, in its most common form, a “reactant compound” within the scope of the present invention may or may not have the reactive moiety(ies) necessary to produce a compound of the present invention. It is intended that such compound(s) will be derivatized to add one or more reactive moiety(ies) by means known to one of ordinary skill in the art. By way of example and not limitation, appropriate derivatives may be produced by hydration, halogenation, carboxylation, amination, nitration, and sulfonation.

“Reaction product” refers to that part of a reactant compound remaining after the chemical reaction producing a covalently-linked compound of the present invention. Such chemical reactions include substitution, elimination, addition, oxidation, and reduction reactions, and involve reactive moieties such as multiple bonds; oxygen and hydroxyl; nitrogen, nitro, amide, and amine; sulfur, sulfhydryl, and sulpho; and other common groups known to one of ordinary skill in the art.

Genetic Linkage Analysis by Mitotic Recombination

The present invention relates to a novel method for genetic linkage analysis by mitotic recombination. By enhancing the production of mitotically-generated recombinant progeny cells, this method will create a large cellular pseudo-family from a single “parent” cell line. Somatic cell recombination will create a loss of the mutant chromosomal locus in a subset of the cellular “progeny”, which will be identified by the loss of the marker for the mutant phenotype. The chromosomal site of recombination will be identified using chromosomal microsatellite markers within these “progeny” cells, which will facilitate a highly focused and targeted positional cloning approach to identify the mutated gene locus in patients having such disorders.

The general application of the inventive approach is dependent on a good cellular/biochemical marker for a disease phenotype. While such markers are not currently available for some diseases, differential gene expression analyses, for example using microarray chips, and rapid screening by reporter gene expression and fluorescence-activated cell sorter (“FACS”) analysis are expected to be effective to identify useful phenotype markers.

Mitotic Recombination

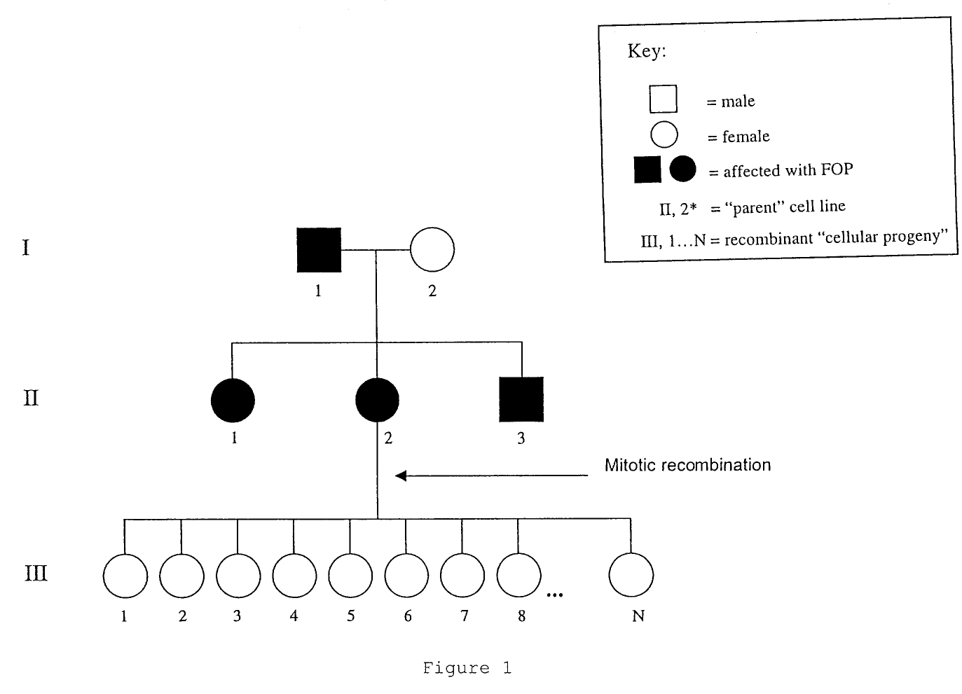

Mitotic recombination (MR) is a reciprocal exchange of genetic material between non-sister chromatids of homologous chromosomes in somatic cells (see FIG. 1). Following mitotic recombination and mitosis, on average, half of the daughter cells will have uniparental isodisomy (the presence of two identical chromosome segments of a single parental origin) for the region distal to the site of recombination.

One consequence of uniparental isodisomy is a phenotypic change as a result of the loss of a dominant allele and the duplication/unmasking of the corresponding recessive allele. Uniparental isodisomy can be detected by the loss of heterozygosity (LOH) of genes and DNA sequence markers that are distal to the recombination site. Applicants use LOH to map the gene for a disorder of interest.

Spontaneous mitotic recombination is rare; the frequency of MR increases in response to a variety of DNA-damaging agents (de Nooij-van Dalen, A. G., van Buuren-van Seggelen, V. H., Lohman, P. H., and Giphart-Gassler, M., Chromosome loss with concomitant duplication and recombination both contribute most to loss of heterozygosity in vitro, Genes, Chromosomes & Cancer 21:30-38 (1998); Holt, D., Dreimanis, M., Pfeiffer, M., Firgaira, F., Morley, A., and Turner, D., Interindividual variation in mitotic recombination, American Journal of Human Genetics 65:1423-1427 (1999); Tischfield, J. A., Loss of heterozygosity or: how I learned to stop worrying and love mitotic recombination, American Journal of Human Genetics 61:995-999 (1997)). The tumor-promoting chemical 12-0-tetradecanolyphorbol-13-acetate (TPA) has been shown to enhance mitotic recombination with a correspondingly low rate of point mutations (Honma, M., and Little, J. B., Recombinagenic activity of the phorbol ester 12-0-tetradecanoylphorbol-13-acetate in human lymphoblastoid cells, Carcinogenesis 16:1717-1722 (1995)). While other agents such as EMS (ethylmethanesulfonate), UV irradiation, and X-rays can induce mitotic recombination, the highest ratio of mitotic recombination to point mutation or chromosome deletions are found with TPA. EMS produces primarily small scale mutations, whereas X-rays and UV irradiation predominantly induce deletions.

Fibrodysplasia Ossificans Progressiva (FOP)

Fibrodysplasia ossificans progressiva (FOP) is the most severe disorder of heterotopic osteogenesis in humans and results in the post-natal formation of an ectopic skeleton. FOP is an extremely rare and disabling autosomal dominant genetic disorder of connective tissue characterized by congenital malformation of the great toes and by progressive disabling heterotopic osteogenesis in characteristic anatomic patterns. Reproductive fitness is low and most new cases arise by spontaneous mutation. Only four small multi-generation families showing autosomal dominant inheritance of FOP have been identified worldwide.

Heterotopic ossification in FOP begins in childhood and can be induced by intramuscular injections. By early adulthood, heterotopic ossification typically leads to ankylosis of all major joints of the axial and appendicular skeleton, rendering movement impossible. Surgical trauma associated with resection of heterotopic bone leads to exacerbation of local ossification. At present, there is no effective prevention or treatment.

Applicants originally hypothesized that over-expression of a bone morphogenetic protein gene may lead to heterotopic ossification in patients with FOP. Applicants expect that BMP4 mRNA and protein are over-expressed in lymphocytes and lesional cells from patients who have FOP and serve as a reliable molecular marker for the condition. Recent studies have indicated that the higher levels of BMP4 mRNA in FOP cells are caused by an increased rate of transcription of the BMP4 gene, and not by an increase in BMP4 mRNA stability. However, the BMP4 gene is not mutated in FOP, and the BMP4 locus on chromosome 14 has been excluded from linkage to the condition, demonstrating the difficulty in identifying the molecular level genetic basis for many genetic disorders.

Recently, a genome-wide linkage analysis using four small families with inheritance of FOP showed linkage (LOD score 3.10 at θ=0) of FOP with 4q27-31, a region that does not include the BMP4 gene, further supporting the conclusion that the FOP causing mutation is not within the BMP4 gene. Genes involved in the BMP signaling pathway, in addition to other candidate genes that may affect BMP4 signaling, have been identified in this interval. Applicants have sequenced genomic DNA from FOP patients for all of the primary candidate genes and no causative mutations have been found.

The FOP linkage interval is a 36 cM region, making identification of the causative gene difficult if it does not present itself as an obvious candidate locus. Additionally, the ability to narrow this region through additional families and/or recombination events is unlikely, given the extreme rarity of this disorder and the low likelihood of ascertaining additional families. Furthermore, since the linkage data was generated using very few families, a measure of uncertainty remains about the reliability of the identified linkage site.

Genetic Linkage Analysis Using MR. Applicants have developed a novel and innovative genetic linkage approach for analyzing rare and not previously characterized genetic disorders. Although the mapping approach is unique, the individual procedures are known to the skilled artisan. The strategy involves the induction of mitotic recombination in a somatic “parent” cell line in order to create a large number of recombinant cellular “progeny”. Mitotic recombination creates a loss of a mutant dominant chromosomal locus in a subset of the “progeny”, a condition that is identified by the loss of a marker for the disease phenotype. As in standard genetic linkage studies, this recombinant subset of the “progeny” that have lost the phenotypic marker are screened with a microsatellite marker panel to correlate the absence of the disease phenotype at the cellular level with the loss of heterozygosity (LOH) of microsatellite haplotypes within a specific chromosomal region.

Applicants' inventive method will be particularly useful for:

a) rare autosomal dominant genetic disorders, for which family pedigrees are scarce and genetic linkage and positional cloning is difficult, inefficient, and/or impossible.

b) disorders caused by defects in different genes in different families (genetic heterogeneity), for which a large cellular family could be generated to obtain statistically significant linkage data for each proband/family independently of other families.

c) genetic disorders for which it is difficult to obtain critical DNA samples from all family members in a pedigree (due to early death from the disorder, for example).

In an example of their novel method, Applicants identify and characterize the mutated gene(s) in a rare disorder of bone cell development, fibrodysplasia ossificans progressiva (FOP). Genetic linkage approaches applied to understanding FOP are expected to be generally applicable to other genetic disorders which have not yet been characterized, whether because of the difficulty, rarity, or simply a relatively lower priority in mapping a particular genetic disorder.

It is expected that the mutated gene(s) in a rare inherited disorder such as fibrodysplasia ossificans progressiva (FOP) can be identified, more quickly and easily than through existing methods, through a loss of heterozygosity linkage analysis in a cellular pseudo-family generated through mitotic recombination of a single parent cell line.

Fibrodysplasia ossificans progressiva (FOP) is an autosomal dominant genetic disorder and is the most disabling form of heterotopic ossification known. FOP is characterized by skeletal malformations of the great toes and by progressive induction of bone formation at ectopic sites. BMP4 mRNA and protein are over-expressed in lymphocytes and lesional cells from patients who have FOP, and BMP4 over-expression is a molecular marker for the condition. The BMP4 gene is not mutated in FOP, and the BMP4 locus has been excluded from linkage to the condition.

FOP is a very rare disorder, and its inheritance from parent to child in families is rare due to low reproductive fitness. Creation of a large, artificial cellular “family” provides an alternate and novel genetic approach to FOP linkage analysis. The strategy involves the induction of mitotic recombination in somatic “parent” cells to create a large number of recombinant cellular “progeny”. To identify the chromosomal region that contains the FOP mutant gene, these progeny are screened for the loss of a marker for the disease phenotype (e.g. BMP4 over-expression) and its correlation with the loss of heterozygosity (LOH) of micro satellite markers for chromosomal loci.

Methods of the Present Invention

The present invention relates to a method for genetic linkage analysis, comprising:

(a) identifying one or more disease phenotype marker(s), each of which is linked to a disease, disorder, or condition of interest;

(b) inducing mitotic recombination in a parent cell line having a mutated gene to produce recombinant progeny cells;

(c) screening said progeny cells for recombinant cellular DNA by selecting those cells having a loss or gain of one or more of said disease phenotype marker(s); and

(d) identifying chromosomal site(s) of recombination within said progeny cells having a loss or gain of disease phenotype marker(s).

In a preferred embodiment, said mitotic recombination in a parent cell line is induced by treatment of a parent cell with 12-0-tetradecanolyphorbol-13-acetate, ethylmethanesulfonate, UV irradiation, X-ray irradiation, or a combination thereof.

In a further preferred embodiment, said mitotic recombination in a parent cell line is induced by treatment of a parent cell with 12-0-tetradecanolyphorbol-13-acetate.

In a further preferred embodiment, said loss of one or more disease phenotype marker(s) is produced by a loss of heterozygosity within said progeny cells.

In a further preferred embodiment, said chromosomal site(s) of recombination within said progeny cells having a loss of disease phenotype marker(s) are identified using chromosomal microsatellite markers.

Said disease phenotype marker is any marker known to artisans of skill in the art. In a preferred embodiment, each said disease phenotype marker is selected from the group consisting of protein over-expression, protein under-expression, expression of a defective protein, lack of protein expression, over-production of mRNA, or under-production of mRNA.

The present invention also relates to a method for identification of a mutated gene, comprising:

(a) identifying one or more disease phenotype marker(s), each of which is linked to a disease, disorder, or condition of interest;

(b) inducing mitotic recombination in a parent cell line having a mutated gene to produce recombinant progeny cells;

(c) screening said progeny cells for recombinant cellular DNA by selecting those cells having a loss or gain of one or more of said disease phenotype marker(s); and

(d) identifying chromosomal site(s) of recombination within said progeny cells having a loss or gain of disease phenotype marker(s);

(e) identifying one or more candidate gene(s) for said mutated gene within said chromosomal site(s) of recombination; and

(f) screening each said candidate gene for mutations.

In a preferred embodiment, said candidate gene(s) are identified by positional cloning.

In a preferred embodiment, said candidate gene(s) are identified by database searching.

In a further preferred embodiment, said screening is achieved by a selection process.

The present invention more particularly relates to a method for genetic linkage analysis, comprising:

(a) identifying one or more disease phenotype marker(s), each of which is linked to a disease, disorder, or condition of interest;

(b) inducing mitotic recombination in a parent cell line having a mutated gene to produce recombinant progeny cells,

wherein said mitotic recombination is induced by induced by treatment of a parent cell with 12-0-tetradecanolyphorbol-13-acetate;

(c) screening said progeny cells for recombinant cellular DNA by selecting those cells having a loss or gain of one or more of said disease phenotype marker(s),

wherein said loss of one or more disease phenotype marker(s) is produced by a loss of heterozygosity within said progeny cells; and

(d) identifying chromosomal site(s) of recombination within said progeny cells having a loss or gain of disease phenotype marker(s),

wherein said chromosomal site(s) of recombination within said progeny cells having a loss of disease phenotype marker(s) are identified using chromosomal microsatellite markers.

The present invention further relates to a method for genetic linkage analysis, comprising:

(a) identifying one or more disease phenotype marker(s), each of which is linked to a disease, disorder, or condition of interest;

(b) inducing mitotic recombination in a parent cell line having a mutated gene to produce recombinant progeny cells,

wherein said mitotic recombination is induced by induced by treatment of a parent cell with 12-0-tetradecanolyphorbol-13-acetate;

(c) screening said progeny cells for recombinant cellular DNA by selecting those cells having a loss or gain of one or more of said disease phenotype marker(s),

wherein said loss of one or more disease phenotype marker(s) is produced by a loss of heterozygosity within said progeny cells;

(d) identifying chromosomal site(s) of recombination within said progeny cells having a loss or gain of disease phenotype marker(s),

wherein said chromosomal site(s) of recombination within said progeny cells having a loss of disease phenotype marker(s) are identified using chromosomal microsatellite markers;

(e) identifying one or more candidate gene(s) for said mutated gene within said chromosomal site(s) of recombination within said progeny cells,

wherein each said candidate gene is identified by positional cloning or database searching; and

(f) screening each said candidate gene for mutations.

Said disease phenotype marker is any marker known to artisans of skill in the art. In a preferred embodiment, each said disease phenotype marker is selected from the group consisting of protein over-expression, protein under-expression, expression of a defective protein, lack of protein expression, over-production of mRNA, or under-production of mRNA.

The present invention further relates to a method for genetic linkage analysis and identification of a mutated gene linked with Fibrodysplasia Ossificans Progressiva, comprising:

(a) inducing mitotic recombination in a parent cell line having a mutated gene linked with Fibrodysplasia Ossificans Progressiva to produce recombinant progeny cells,

wherein said mitotic recombination is induced by induced by treatment of a parent cell with 12-0-tetradecanolyphorbol-13-acetate;

(b) screening said progeny cells for recombinant cellular DNA by selecting those cells having a loss of BMP4 over-expression, loss of BMP antagonist response to a BMP4 stimulus, or decrease in the density of BMP receptor BMPRIA,

wherein said loss of BMP4 over-expression, loss of BMP antagonist response to a BMP4 stimulus, or decrease in the density of BMP receptor BMPRIA is produced by a loss of heterozygosity within said progeny cells;

(c) identifying chromosomal site(s) of recombination within said progeny cells having a loss of BMP4 over-expression, loss of BMP antagonist response to a BMP4 stimulus, or decrease in the density of BMP receptor BMPRIA,

wherein said chromosomal site(s) of recombination within said progeny cells having a loss of BMP4 over-expression, loss of BMP antagonist response to a BMP4 stimulus, or decrease in the density of BMP receptor BMPRIA are identified using chromosomal microsatellite markers;

(d) identifying one or more candidate gene(s) for said mutated gene linked with Fibrodysplasia Ossificans Progressiva within said chromosomal site(s) of recombination within said progeny cells,

wherein each said candidate gene is identified by positional cloning or database searching; and

(e) screening each said candidate gene for mutations.

The present invention further relates to a method for treating Fibrodysplasia Ossificans Progressiva, comprising:

(a) identifying a defective gene responsible for causing Fibrodysplasia Ossificans Progressiva;

(b) harvesting somatic parent cells from an individual having a mutated gene producing the phenotype for Fibrodysplasia Ossificans Progressiva;

(c) producing recombinant progeny cells by a method of inducing mitotic recombination in said parent cells line having a mutated gene;

(d) screening said recombinant progeny cells for recombinant cells having a wild-type copy of said defective gene; and

(e) re-introducing said recombinant cells having a wild-type copy of said defective gene into said individual.

In a preferred embodiment, said defective gene responsible for causing Fibrodysplasia Ossificans Progressiva is identified by a method described above.

The present invention further relates to a method for treating Fibrodysplasia Ossificans Progressiva in an individual in need of thereof, comprising:

(a) identifying a defective gene responsible for causing Fibrodysplasia Ossificans Progressiva; and

(b) administering a composition which upregulates the expression of a wild-type gene, down regulates the expression of said defective gene, or indirectly sequesters or deactivates a defective protein produced by expression of said defective gene.

In a preferred embodiment, said defective gene responsible for causing Fibrodysplasia Ossificans Progressiva is identified by a method described above.

In a further preferred embodiment, said composition for upregulation of the expression of a wild-type gene is a vector encoding, or a delivery system carrying, one or more copies of an isolated exogenous wild-type copy of said defective gene which is capable of being expressed in said individual.

In a further preferred embodiment, said vector or delivery system is selected from the group consisting of an adenovirus vector, a lentivirus vector, a lentiviral/adenoviral chimeric vector, and a liposome packaging and delivery system.

In a further preferred embodiment, said vector is a replication-defective adenovirus vector.

Kits and Apparatus of the Present Invention

The present invention further relates to a kit for producing a clonally-expanded, nested cellular progeny cell set from a parent cell, comprising:

(a) a means for obtaining and isolating a parent cell line from an individual;

(b) a means for inducing mitotic recombination in said parent cell line to produce progeny cells;

(c) a means for growing said progeny cells; and

(d) a means for screening said progeny cells for recombinant progeny cells.

In a preferred embodiment, said means for inducing mitotic recombination is selected from the group consisting of 12-0-tetradecanolyphorbol-13-acetate, ethylmethanesulfonate, UV irradiation, X-ray irradiation, and a combination thereof.

In a further preferred embodiment, said means for inducing mitotic recombination comprises 12-0-tetradecanolyphorbol-13-acetate.

The present invention further relates to an apparatus for genetic linkage analysis of a cell of an organism having a genetic mutation of interest and a linked disease phenotype marker, comprising:

(a) a first stage comprising a system for inducing mitotic recombination in a parent cell line to produce clonally-expanded progeny cells;

(b) a second stage comprising a cell sorting system for selection for the loss or gain of expression of said disease phenotype marker;

(c) a third stage comprising an antibiotic selection system for selection of cells that contain said disease phenotype marker;

(d) a fourth stage comprising an isolation system for isolating genomic DNA from said progeny cells;

(e) a fifth stage comprising a screening system for screening said genomic DNA for one or more genomic marker(s); and

(f) a sixth stage comprising a system for correlating said sites of homologous recombination and loss or gain of said mutant phenotype with the genomic DNA sequence of said organism.

In a preferred embodiment, said means for inducing mitotic recombination is selected from the group consisting of 12-0-tetradecanolyphorbol-13-acetate, ethylmethanesulfonate, UV irradiation, X-ray irradiation, and a combination thereof.

In a further preferred embodiment, said means for inducing mitotic recombination comprises 12-0-tetradecanolyphorbol-13-acetate.

In a further preferred embodiment, said isolation system comprises proteinase K, SDS, and phenol.

In a further preferred embodiment, said screening system comprises a PCR-based technique using microsatellite markers.

In a further preferred embodiment, said system for correlating is a human genomic database maintained on one or more computers.

Nucleic Acid Sequences of the Present Invention The present invention also relates to a DNA sequence containing a mutation, identified by a process comprising:

(a) inducing mitotic recombination in a parent cell line having a mutated gene linked with Fibrodysplasia Ossificans Progressiva to produce recombinant progeny cells,

wherein said mitotic recombination is induced by induced by treatment of a parent cell with 12-0-tetradecanolyphorbol-13-acetate;

(b) screening said progeny cells for recombinant cellular DNA by selecting those cells having a loss of BMP4 over-expression, loss of BMP antagonist response to a BMP4 stimulus, or decrease in the density of BMP receptor BMPRIA,

wherein said loss of BMP4 over-expression, loss of BMP antagonist response to a BMP4 stimulus, or decrease in the density of BMP receptor BMPRIA is produced by a loss of heterozygosity within said progeny cells;

(c) identifying chromosomal site(s) of recombination within said progeny cells having a loss of BMP4 over-expression, loss of BMP antagonist response to a BMP4 stimulus, or decrease in the density of BMP receptor BMPRIA,

wherein said chromosomal site(s) of recombination within said progeny cells having a loss of BMP4 over-expression, loss of BMP antagonist response to a BMP4 stimulus, or decrease in the density of BMP receptor BMPRIA are identified using chromosomal microsatellite markers;

(d) identifying one or more candidate gene(s) for said mutated gene linked with Fibrodysplasia Ossificans Progressiva within said chromosomal site(s) of recombination within said progeny cells,

wherein each said candidate gene is identified by positional cloning or database searching;

(e) sequencing each said candidate gene;

(f) comparing the DNA sequence for each candidate gene with a wild-type DNA sequence of said gene; and

(g) identifying said DNA sequence containing a mutation for each said candidate gene.

Route(s) of Administration

In a preferred embodiment of the methods for treating Fibrodysplasia Ossificans Progressiva, isolated wild-type DNA for each gene identified by the method for genetic linkage analysis and identification of a mutated gene linked with Fibrodysplasia Ossificans Progressiva is inserted into an appropriate vector readily identifiable by one of ordinary skill in the art and described further herein. The vector is introduced into the host by methods readily identifiable by one of ordinary skill in the art and described further herein.

Alternate route(s) of administration of the compositions of the present invention are well known to those skilled in the art (see, for example, “Remington's Pharmaceutical Sciences”, 18th Edition, Chapter 86, pp. 1581-1592, Mack Publishing Company, 1990). The compositions may be administered orally, parenterally, by inhalation spray, topically, rectally, nasally, buccally, vaginally, or via an implanted reservoir in dosage formulations containing conventional non-toxic pharmaceutically-acceptable carriers, adjuvants, and vehicles. The term parenteral as used herein includes subcutaneous, intravenous, intramuscular, intraperitoneally, intrathecally, intraventricularly, intrasternal, and intracranial injection or infusion techniques.

The compounds and compositions may be administered in the form of sterile injectable preparations, for example, as sterile injectable aqueous or oleaginous suspensions. These suspensions, may be formulated according to techniques known in the art using suitable dispersing or wetting agents and suspending agents. The sterile injectable preparations may also be sterile injectable solutions or suspensions in non-toxic parenterally-acceptable diluents or solvents, for example, as solutions in 1,3-butanediol. Among the acceptable vehicles and solvents that may be employed are water, Ringer's solution and isotonic sodium chloride solution. In addition, sterile, fixed oils are conventionally employed as solvents or suspending mediums. For this purpose, any bland fixed oil such as a synthetic mono- or di-glyceride may be employed. Fatty acids such as oleic acid and its glyceride derivatives, including olive oil and castor oil, especially in their polyoxyethylated versions, are useful in the preparation of injectables. These oil solutions or suspensions may also contain long-chain alcohol diluents or dispersants.

Additionally, the compounds and compositions may be administered orally in the form of capsules, tablets, aqueous suspensions, or solutions. Tablets may contain carriers such as lactose and corn starch, and/or lubricating agents such as magnesium stearate. Capsules may contain diluents including lactose and dried corn starch. Aqueous suspensions may contain emulsifying and suspending agents combined with the active ingredient. The oral dosage forms may further contain sweetening, flavoring, coloring agents, or combinations thereof. Delivery in an enterically coated tablet, caplet, or capsule, to further enhance stability and provide release in the intestinal tract to improve absorption, is the best mode of administration currently contemplated.

The compounds and compositions may also be administered rectally in the form of suppositories. These compositions can be prepared by mixing the drug with a suitable non-irritating excipient which is solid at room temperature, but liquid at rectal temperature and, therefore, will melt in the rectum to release the drug. Such materials include cocoa butter, beeswax, and polyethylene glycols.

Furthermore, the compounds may be administered topically, especially when the conditions addressed for treatment involve areas or organs readily accessible by topical application, including the lower intestinal tract. Suitable topical formulations can be readily prepared for such areas or organs. For example, topical application to the lower intestinal tract can be effected in a rectal suppository formulations (see above) or in suitable enema formulations.

It is envisioned that the continuous administration or sustained delivery of the compounds and compositions of the present invention may be advantageous for a given condition. While continuous administration may be accomplished via a mechanical means, such as with an infusion pump, it is contemplated that other modes of continuous or near continuous administration may be practiced. For example, such administration may be by subcutaneous or muscular injections as well as oral pills.

Techniques for formulating a variety of other sustained- or controlled-delivery means, such as liposome carriers, bio-erodible particles or beads and depot injections, are also known to those skilled in the art.

Dosage

The novel methods of the invention involve administration of a therapeutically effective amount of the active agent indicated above. This effective amount can vary depending upon the physical status of the patient and other factors well known in the art. Moreover, it will be understood that this dosage of active agent can be administered in a single or multiple dosage units to provide the desired therapeutic effect. If desired, other therapeutic agents can be employed in conjunction with those provided by the present invention. The compounds of the invention are preferably delivered to the patient by means which are well known in the art. Solid form pharmaceutical preparations which may be prepared according to the present invention include powders, tablets, dispersible granules, capsules, cachets and suppositories. In general, solid form preparations will comprise from about 5% to about 90% by weight of the active agent.

A solid carrier can be one or more substances which may also act as diluents, flavoring agents, solubilizers, lubricants, suspending agents, binders or tablet disintegrating agents; it can also be encapsulating material. In powders, the carrier is a finely divided solid which is in admixture with the viscous active compound. In tablets, the active compound is mixed with a carrier having the necessary binding properties in suitable proportions and compacted to the shape and size desired. Suitable solid carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation is intended to include the formulation of the active compound with encapsulating materials as a carrier which may provide a capsule in which the active component (with or without other carriers) is surrounded by carrier, which is thus in association with it. Similarly, cachets are included. Tablets, powders, cachets, and capsules can be used as solid dosage forms suitable for oral administration. If desired for reasons of convenience or patient acceptance, pharmaceutical tablets prepared according to the invention may be provided in chewable form, using techniques well known in the art.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby to solidify.

Liquid form preparations include solutions, suspensions, and emulsions. As an example may be mentioned water or water/propylene glycol solutions for parenteral injection. Liquid preparations can also be formulated in solution in aqueous polyethylene glycol solution. Aqueous solutions suitable for oral use can be prepared by dissolving the active component in water and adding suitable colorants, flavors, stabilizers and thickening agents as desired. Aqueous suspensions suitable for oral use can be made my dispersing the finely divided active component in water with a viscous material, i.e., natural or synthetic gums, resins, methylcellulose, sodium carboxymethylcellulose, and other well known suspending agents. Liquid pharmaceutical preparations may comprise up to 100% by weight of the subject active agent.

Also contemplated as suitable carriers are solid form preparations which are intended to be converted, shortly before use, to liquid form preparations for either oral or parenteral administration. Such liquid forms include solutions, suspensions, and emulsions. These particular solid form preparations are most conveniently provided in unit dose form and as such are used to provide a single liquid dosage unit. Alternately, sufficient solid may be provided so that after conversion to liquid form, multiple individual liquid doses may be obtained by measuring predetermined volumes of the liquid form preparation as with a syringe, teaspoon, or other volumetric container. When multiple liquid doses are so prepared, it is preferred to maintain the unused portion of said liquid doses at low temperature (i.e., under refrigeration) in order to retard possible decomposition. The solid form preparations intended to be converted to liquid form may contain, in addition to the active material, flavorants, colorants, stabilizers, buffers, artificial and natural sweeteners, dispersants, thickeners, solubilizing agents, and the like. The liquid utilized for preparing useful liquid form preparations may be water, isotonic water, ethanol, glycerine, propylene glycol, and the like as well as mixtures thereof. Naturally, the liquid utilized will be chosen with regard to the route of administration. For example, liquid preparations containing large amounts of ethanol are not suitable for parenteral use.

The pharmaceutical preparation may also be in a unit dosage form. In such form, the preparation is subdivided into unit doses containing appropriate quantities of the active component. The unit dosage form can be a packaged preparation, the package containing discrete quantities of preparation, for example, packeted tablets, capsules, and powders in vials or ampoules. The unit dosage form can also be a capsule, cachet, or tablet itself or it can be the appropriate number of any of these in packaged form.

The pharmaceutical preparations of the invention may include one or more preservatives well known in the art, such as benzoic acid, ascorbic acid, methylparaben, propylparaben and ethylenediaminetetraacetic acid (EDTA) Preservatives are generally present in amounts up to about 1% and preferably from about 0.05 to about 0.5% by weight of the pharmaceutical composition.

Useful buffers for purposes of the invention include citric acid-sodium citrate, phosphoric acid-sodium phosphate, and acetic acid-sodium acetate in amounts up to about 1% and preferably from about 0.05 to about 0.5% by weight of the pharmaceutical composition. Useful suspending agents or thickeners include cellulosics like methylcellulose, carageenans like alginic acid and its derivatives, xanthan gums, gelatin, acacia, and microcrystalline cellulose in amounts up to about 20% and preferably from about 1% to about 15% by weight of the pharmaceutical composition.

Sweeteners which may be employed include those sweeteners, both natural and artificial, well known in the art. Sweetening agents such as monosaccharides, disaccharides and polysaccharides such as xylose, ribose, glucose, mannose, galactose, fructose, dextrose, sucrose, maltose, partially hydrolyzed starch or corn syrup solids and sugar alcohols such as sorbitol, xylitol, mannitol and mixtures thereof may be utilized in amounts from about 10% to about 60% and preferably from about 20% to about 50% by weight of the pharmaceutical composition. Water soluble artificial sweeteners such as saccharin and saccharin salts such as sodium or calcium, cyclamate salts, acesulfame-K, aspartame and the like and mixtures thereof may be utilized in amounts from about 0.001% to about 5% by weight of the composition.

Flavorants which may be employed in the pharmaceutical products of the invention include both natural and artificial flavors, and mints such as peppermint, menthol, vanilla, artificial vanilla, chocolate, artificial chocolate, cinnamon, various fruit flavors, both individually and mixed, in amounts from about 0.5% to about 5% by weight of the pharmaceutical composition.

Colorants useful in the present invention include pigments which may be incorporated in amounts of up to about 6% by weight of the composition. A preferred pigment, titanium dioxide, may be incorporated in amounts up to about 1%. Also, the colorants may include other dyes suitable for food, drug and cosmetic applications, known as F.D.&C. dyes and the like. Such dyes are generally present in amounts up to about 0.25% and preferably from about 0.05% to about 0.2% by weight of the pharmaceutical composition. A full recitation of all F.D.&C. and D.&C. dyes and their corresponding chemical structures may be found in the Kirk-Othmer Encyclopedia of Chemical Technology, in Volume 5, at pages 857-884, which text is accordingly incorporated herein by reference.

Useful solubilizers include alcohol, propylene glycol, polyethylene glycol and the like and may be used to solubilize the flavors. Solubilizing agents are generally present in amounts up to about 10%; preferably from about 2% to about 5% by weight of the pharmaceutical composition.

Lubricating agents which may be used when desired in the instant compositions include silicone oils or fluids such as substituted and unsubstituted polysiloxanes, e.g., dimethyl polysiloxane, also known as dimethicone. Other well known lubricating agents may be employed.

It is not expected that compounds of the present invention will display significant adverse interactions with other synthetic or naturally occurring substances. Thus, a compound of the present invention may be administered in combination with other compounds and compositions of the present invention and other active agents as disclosed herein.

The optimal pharmaceutical formulations will be determined by one skilled in the art depending upon considerations such as the route of administration and desired dosage. See, for example, “Remington's Pharmaceutical Sciences”, 18th ed. (1990, Mack Publishing Co., Easton, Pa. 18042), pp. 1435-1712, the disclosure of which is hereby incorporated by reference. Such formulations may influence the physical state, stability, rate of in vivo release, and rate of in vivo clearance of the present therapeutic agents of the invention.

The amount of active ingredient that may be combined with the carrier materials to produce a single dosage form will vary depending upon the host treated and the particular mode of administration. It is understood, however, that a specific dose level for any particular patient will depend upon a variety of factors, including the activity of the specific compound employed; the age, body weight, general health, sex and diet of the patient; the time of administration; the rate of excretion; drug combination; the severity of the particular disorder being treated; and the form of administration. One of ordinary skill in the art would appreciate the variability of such factors and would be able to establish specific dose levels using no more than routine experimentation.

### EXAMPLES

The following examples are illustrative of the present invention and are not intended to be limitations thereon. Unless otherwise indicated, all percentages are based upon 100% by weight of the final composition.

### Example 1

Selection of a Source of Cells for “Parent” Cell Line

Applicants' preferred procedures for genetic linkage analysis by mitotic recombination begins with the selection of a parent cell line. Any patient with a genetic disorder is a potential candidate for selection as a source for a parent cell line. However, a patient with at least a second-generation genetic disorder is predicted to have a germline mutation and therefore is expected to carry the mutation in all cells (i.e. no chance of being a somatic cell mosaic). Therefore, cells from at least second generation patients are the preferred choice as the source for the parent cell line in the inventive methods. Additionally, a selected cell line preferably has high expression levels of the marker for a mutant phenotype. The selected parent cell line is preferably derived from a female subject, since higher natural recombination rates have been observed in female vs. male cells.

Exemplary Methods in FOP

The exemplary method in FOP uses cells from a patient with the inherited disorder fibrodysplasia ossificans progressive (FOP). FOP is a suitable example because: (a) a reliable cellular assay for the FOP phenotype (high expression levels of BMP4 mRNA) is available; and (b) FOP is an autosomal dominant disorder, therefore the loss of the mutant allele through recombination will revert the cell to a “normal” phenotype. Applicants' method creates a cellular “pseudo-family” that can be used in genetic studies to identify the cause of an inherited disease, disorder, or condition.

Patients that are optimal sources of “parent” cells are affected daughters in a second generation FOP family. Such patients' lymphoblastoid cell lines (“LCLs”) are evaluated for BMP4 expression by a reporter gene driven by the BMP4 promoter. A parent cell line is transfected with a reporter construct for the FOP phenotype that carries a selectable marker (antibiotic resistance) and a reporter gene such as green fluorescent protein (GFP) that is regulated by the BMP4 promoter.

Following transfection, cells are grown in the presence of antibiotic to propagate only cells carrying the selection/reporter vector. As with any selectable marker, some cells will resist negative selection despite the lack of a resistance gene. Therefore, the GFP reporter is also used as a sorting marker to recover viable cells in FACS analysis, providing an efficient means to exclude all non-transfected cells. Only cells carrying and expressing the selection/reporter vector are selected for further studies, ensuring that essentially all progeny cells generated carry the vector.

Selection/reporter constructs. Human BMP4 promoter sequences are subcloned using KpnI-XhoI sites into a mammalian expression vector with a bioluminescent reporter gene such as GFP. The vectors also carry a selection gene for neomycin resistance and a multiple cloning site. Standard cloning and plasmid growth methods, as known to those of ordinary skill in the art, are used.

Antibiotic selection. Prior to transfection with the reporter/selection vector, a dose-response of the “parent” cell line to antibiotic treatment is determined. This will establish an optimal culturing time and dose during selection for transfected cells. Cells are treated with 100-800 μg/ml of antibiotic for 2-4 weeks, keeping in mind that a multiplying cell requires 3-7 days for death to occur in the presence of antibiotic. Cell viability is checked using Trypan blue staining.

Cell transfection. Plasmid DNAs for transfection are purified using known, recommended protocols and DNA concentrations are determined, preferably fluorometrically. It is expected that previous experiments have optimized the transfection efficiency for DNA concentration, LCL density, and time of transfection. 1 μg of selection/reporter plasmid DNA per 2.5×10⁶ LCLs are transfected with a cationic lipid transfection reagent, such as LipofectAMINE Plus reagent, in RPMI (Roswell Park Memorial Institute) medium without serum or antibiotics following standard protocols.

After 16 hours, fresh media containing serum and antibiotics is added to the cells. Transfected LCLs are cultured in the optimal dose of antibiotic for 2 weeks or more to select against non-transfected cells and ensure stable transfection. Both the “parent” cell line and a control non-mutant cell line are transfected with the construct.

Flow cytometry. Fluorescence profiles for untransfected LCLs and LCLs transfected with the reporter (GFP) vector with no BMP4 promoter are used to determine background auto-fluorescence of the LCLs. Flow cytometry is used to identify and recover transfected control and “parent” cells that contain the selection/reporter construct (i.e. express the GFP reporter gene), and the population is expanded prior to TPA treatment. Transfected control and FOP LCLs are compared for relative GFP signals.

Experimental Alternatives. Applicants' inventive methods are greatly facilitated by the use of a phenotypic marker that can be screened for easily and rapidly by FACS analysis. Mammalian expression vectors with several different bioluminescent reporter genes are available, as well as enhanced variants, which are more highly expressed in mammalian cells and have a higher fluorescence intensity, are available and preferred. Fluorescent proteins are readily detected by flow cytometry. Efficiency of selection and reduced background may be improved by co-transfection with a second antibiotic (for example, hygromycin) selection/reporter construct. Electroporation to transfect cells is an option to increase the rate of co-transfection. If a second reporter is needed, one with an excitation and emission range easily distinguished from GFP is preferred.

The selection/reporter DNA constructs can be introduced into the “parent” cells prior to TPA treatment or into “progeny” cells after treatment. Transfecting cells before the TPA treatment has the advantage that only cells that stably carry and express the selection/reporter genes are selected for treatment with TPA, therefore ensuring that all (or at least a very high percentage) of recombinant “progeny” cells will contain these markers, making the transfection efficiency less of a critical consideration.

If the selection/reporter constructs are transfected into “progeny” cells, only a fraction of the cells will contain the constructs, and a substantially greater number of progeny cells would need to be generated in order to obtain the required number for analysis. Introduction of the construct into the cellular progeny is an alternative (although more labor intensive) option.

Plasmid or viral vectors can be used for the selection/reporter construct. Plasmids offer advantages in ease of handling and therefore are the preferred vector in Applicants' methods. Transfection efficiency of plasmids into LCLs is expected to be about 5%. This does not present difficulties if the “parent” cells are transfected and selected for the selection/reporter construct prior to TPA treatment. In the event that transfection is done following TPA treatment, adenovirus or retrovirus constructs can be used as the alternative vector. The generation of viral constructs is known to the ordinarily skilled artisan.

Cells that grow as monolayers in culture are easier to grow and transfect; therefore cell lines having such characteristic are preferred. However, such cell lines may not always be available, and alternative target cell lines may be utilized.

Expected Results. FIG. 1 depicts an overview of the methods of the present invention for generation of a “parent” cell line for producing a genetic linkage map by mitotic recombination. Generation I consists of an affected father and unaffected mother; generation II consists of multiple second-generation affected children, wherein the preferred “parent” cell line is derived from the cells of an affected daughter of the generation I parents; and generation III consists of a large cellular pseudo-family produced by mitotic recombination using Applicants' inventive method.

### Example 2

Marker(s) for a Mutant Phenotype

Induction of recombination in somatic “parent” cells produces both sister and non-sister chromatid exchange. When mitotic recombination exchanges a mutant allele for a normal allele, in one embodiment a subset of “progeny” cells are generated which revert to a “normal” phenotype. The same result is expected when recombination of said marker produces progeny cells having a mutant phenotype for said marker. The preferred embodiments of the present invention depend on the availability of a reliable marker for the presence and loss of the mutant phenotype. The location of the mutated locus can then be identified in a subsequent process by the loss of a marker linked to the disease gene.

Exemplary Methods in FOP

Extensive data generated by RT-PCR, Northern blots, and/or RNase protection assays demonstrate that increased expression of BMP4 mRNA is a reliable cellular marker for FOP. A high level of BMP4 mRNA expression is a reliable and invariant phenotypic marker for FOP; a loss of the chromosomal region that carries the mutant FOP allele results in a severe reduction in BMP4 transcription in these cells, which is monitored by expression of GFP from a BMP4 promoter.

After transfection with selection/reporter constructs (Example 1), cells from the parent cell line are treated with 12-0-tetradecanolyphorbol-13-acetate (TPA) to enhance the rate of mitotic recombination in these cells. For lymphoblastoid cell lines, the de novo mitotic recombination rate (MRR) is approximately 10⁻⁶ per cell per generation, which is increased to approximately 10⁻⁵ with TPA treatment.

Applicants expect that ˜200×10⁶ cells are to be treated with TPA to recover ˜5-10 cells with recombination on the chromosome that carries the FOP mutant allele. To screen for LOH at the FOP locus, the GFP reporter under the regulation of the BMP4 promoter is used as a sorting marker in FACS analysis. Antibiotic selection is maintained to verify that lack of BMP4 reporter expression is not due to loss of the reporter construct. Progeny with expression of the BMP4 reporter (i.e. those which have lost the FOP phenotype) are predicted to have lost the genetic locus containing the mutant FOP gene through homologous mitotic recombination, and are analyzed for the chromosomal site of recombination.

Cell cultures. Lymphoblastoid cell lines (LCLs) are grown in RPMI 1640 media with penicillin/streptomycin/fungizone and 15% fetal calf serum. The cells are maintained in suspension culture at cell densities ranging from 1-10×10⁵ cells/ml.

Estimation of the number of cells to be screened. TPA treatment is expected to produce one recombination event per 10⁵ cells. For one recombination event per chromosome arm [(18×2)+(5×1)=41 arms; excluding short arms of the five acrocentric chromosomes], ˜4×10⁶ cells are needed. However, to compensate for varying chromosome size (˜300 cM for chromosome 1 to ˜75 cM for chromosome 22, a 4 to 5 fold difference), the number of cells required increases to 20×10⁶ cells. Since it is expected that only 50% of recombinants in the region of interest will demonstrate LOH, the number of cells required becomes 40×10⁶ cells. To ensure that multiple cells per recombined chromosome are recovered, at least 200 to 400×10⁶ cells are used to generate the desired cellular pseudo-family. TPA induction of batches of 200×10⁶ cells are repeated to obtain sufficient numbers of recombinants, as needed.

TPA treatment. The “parent” cell lines selected for analysis are tested for cytotoxicity, and the highest tolerated dose, as determined by growth curves, is selected. Homologous recombination is then induced using 12-0-tetradecanolyphorbol-13-acetate either at the known effective dose rate of 1-10 μg/ml or preferably at the highest dose tolerated in the absence of major toxicity. A control group with no TPA treatment is maintained for comparison.

Cultures up to 50 ml (˜4×10⁵ cells/ml) are treated with TPA using a stock solution of 5 mg/ml in 0.1% acetone (prepared fresh before each use). Cells are treated for 24 hours, then recovered by centrifugation, washed twice with 10 ml phosphate-buffered saline (PBS), and resuspended in fresh culture medium. Cultures are grown in non-selective medium for 1-3 days to allow expression of antibiotic resistance prior to addition of the selection medium. The dose response to antibiotic resistance/selection in each cell line is pre-determined in non-transfected cells.

Flow cytometry. Fluorescence profiles for FACS markers are generated to determine background fluorescence. Following treatment of the parent cells with TPA, the cellular progeny are analyzed by flow cytometry to identify and recover cells that have lost reporter expression (i.e. cells that have lost the FOP mutant allele). A sort of ˜200×10⁶ cells will take 5-6 hours. Transfected LCLs from the unaffected mother of the source of the parent cells are used as a negative control to determine fluorescent background in order to properly calibrate the cell-sorter.

Expansion of selected cellular progeny. Selected cells from the FACS sort are grown under antibiotic selection until populations of 10-50×10⁶ cells (enough for DNA/RNA isolation) are obtained. An aliquot of these cells is frozen for future analyses.

Experimental Alternatives. The mitotic recombination rate (MRR) varies considerably among individual cell lines. Preferably, multiple cell lines are used in the inventive methods; in this regard, at least two cell lines are preferred. If one or more cell line(s) demonstrate a higher recombination rate in a first round of experiments, then it/they is/are used in all subsequent experiments. Data from multiple cell lines can be pooled, however, when multiple cell lines are generated from individuals, such as siblings, who carry the identical genetic mutation.

Lymphocytes typically perform well in high speed sorting, with speeds up to 10,000 cells per second expected. Immediate resorting of cells is difficult but the population can be sorted after reculturing. Culturing cells following a sort can yield low recovery rates. This potential problem may be partially ameliorated by using a co-culture technique.

Addressing theoretical false positives. (a) With any selectable marker, some cells will resist negative selection despite the lack of a resistance gene. In the event that neomycin resistance is not sufficiently stringent, hygromycin resistance gene vectors are substituted. If even greater selectivity is required, co-transfection with a second vector construct that includes a second antibiotic resistance gene places additional selection on the cells and reduce the chances of escape resistance. Additionally, a second vector contains a constitutively expressed marker or a second reporter under the control of the BMP4 promoter.

(b) Random point mutations at loci other than the one responsible for FOP (i.e. genes in the BMP4 pathway that disrupt BMP signaling) are an alternative explanation for loss of mutant phenotype. However, such point mutations are random and any such progeny cells are expected to be easily distinguished from the predominant LOH set by lack of the fluorescence marker and are discarded as background.

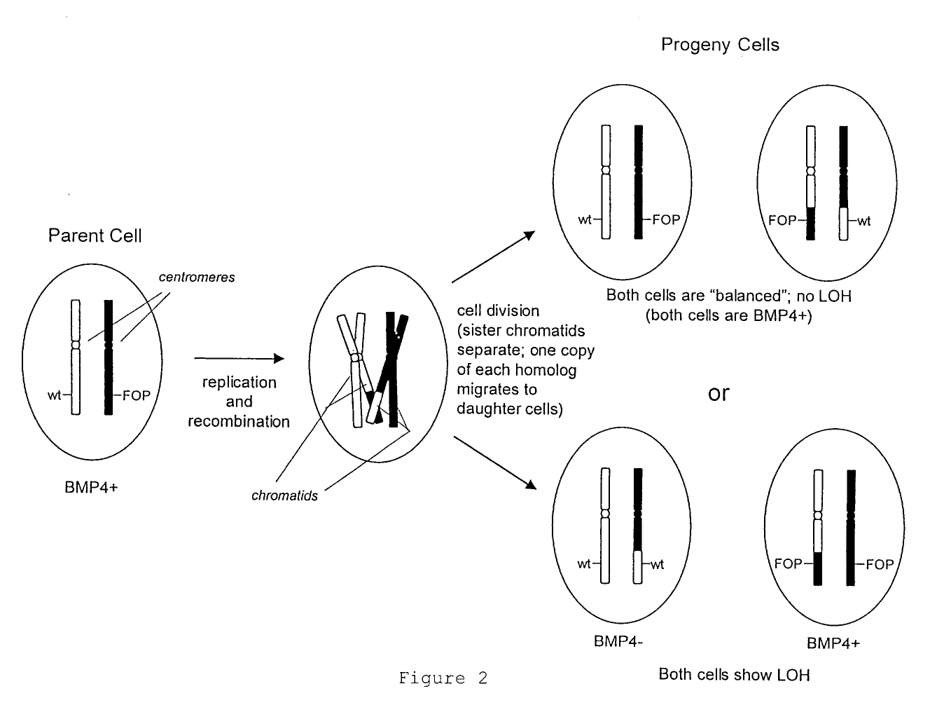

Expected Results. Referring to FIG. 2, a “parental” cell from an FOP patient is shown at left with a representational pair of homologous chromosomes containing the gene locus for FOP. Since FOP is an autosomal dominant disorder, cells from FOP patients carry one mutant copy of the gene (“FOP”) and one normal copy of the gene (wild type, or “wt”). In the case of the FOP family described in FIG. 1, it is known that the mutated allele has been paternally inherited (dark chromosome and centromere). The normal copy of the gene is carried on the maternally inherited chromosome homolog (light chromosome and centromere). The phenotype of the cells when one copy of the mutated FOP gene is present is over-expression of BMP4 (“BMP4+”). Following DNA replication, each duplicated chromosome homolog consists of two chromatids. The DNA sequences of each chromatid of a duplicated chromosome are identical. However, homologous recombination between chromatids by reciprocal exchange of chromosome segments is induced by treatment with TPA. This is indicated in the figure by the exchange of segments of a dark and light chromatid. During mitotic cell division, one chromatid from each of the two chromosome homologs will separate into progeny cells. The chromosome homolog identity is determined at the centromere region, therefore, the easiest way to track chromosomes in the figure is by noting the dark or light centromere. Each chromatid with a light centromere can segregate into progeny cells with either of the two chromatids with dark centromeres (and vice versa). Therefore, following a recombination event, there is an equal chance that either of two sets of daughter cells will result:

One possibility (at top right of the figure) is that both progeny cells will be “balanced” i.e. they will contain all of the same chromosome/DNA material that is present in the parental cell (although the organization of this material has been altered by recombination in one of those cells). There is no loss of heterozygosity (LOH) in either cell with regard to the mutant FOP locus: each progeny cell contains one normal and one FOP allele, and both will continue to over-express BMP4.

The second possibility (at bottom right of figure) results in daughter cells that contain chromosome/DNA segments that are derived from only one parental homolog. In the example shown, one cell would contain two copies of the mutated FOP gene while the other would contain two copies of the normal allele. Both cells would show loss of heterozygosity for the mutant FOP locus. The cell with two normal alleles would lose the FOP mutant phenotype (“BMP4−”)

### Example 3

Characterization of the Human BMP4 Promoter Expression in FOP and Control LCLs

The progeny that lose a mutant allele through mitotic recombination will revert to a “normal” phenotype, as determined by expression of a reporter gene. In an alternate embodiment, the progeny that lose a mutant allele through mitotic recombination will display a “mutant” phenotype, as determined by expression of a reporter gene. Similar to the approach used by standard genetic linkage studies, these mitotically recombinant and clonally-expanded “cellular progeny” are screened using polymorphic microsatellite markers. The loss of the disease phenotype of the cells is correlated with loss of heterozygosity (LOH) of the microsatellite alleles. The recombinant progeny that lose the mutant phenotype are expected to show LOH for the microsatellite markers that are distal to a recombination site that has exchanged the chromosomal region containing the mutant allele for a wild-type allele.

Genomic DNA is isolated from clonally-expanded cellular “progeny” that have been selected for the loss of expression from the reporter and that have been verified to contain the reporter construct by antibiotic selection. A preliminary analysis of the “parent” cell line establishes that all analyzed microsatellite markers are heterozygous. Using standard methods, preferably PCR-based techniques, known to those of skill in the art, the isolated DNAs are screened with microsatellite markers. The microsatellite marker alleles in the “progeny” cells are evaluated for LOH as compared to the “parent” cell. Assuming that only a single recombination event occurs on a given chromosome, it is only necessary to monitor a single telomeric marker per chromosome arm, a total of 41 markers excluding acrocentric chromosome arms. The most distal heterozygous marker for each arm that is identified in the parent cell is selected for this analysis.

All of the cells that have lost marker expression are expected to have the same chromosome marker showing LOH. A more specific localization of the mutant gene locus is then made by “fine mapping” with additional markers along that chromosome arm. The chromosomal region containing the mutant gene locus is identified by the most distal recombinant in a nested set of loci showing marker LOH. RT-PCR for marker mRNA expressed from the cells is used to confirm the loss of the mutant phenotype, indicated by marker expression, in the progeny cells that do not express the reporter construct.

Exemplary Methods in FOP

A nested set of BMP4 gene 5′ flanking sequences have been examined for regulation of a luciferase reporter gene. The initial assays in U-2 OS cells showed the highest level of promoter activity with a ˜2 kb segment of the BMP4 promoter. Transfection of the nested set of reporter constructs into FOP and control LCLs indicate that LCLs and U-2 OS cells utilize the same promoter regions for positive and negative regulation of BMP4 transcription. Comparison of BMP4 promoter activity in FOP and control LCLs showed 8-10 fold higher normalized expression levels in FOP cells. This level of difference is expected to be readily resolved by FACS analysis.

Cell culture. Each selected “progeny” LCL is expanded in RPMI 1640 media with antibiotics and serum. It is expected that ˜400 μg of genomic DNA can be recovered from 50×10⁶ LCLs. Approximately 40 μg of DNA are needed for a genome-wide analysis with microsatellite markers and about 100 μg of DNA from each “progeny” is isolated. About 25 μg RNA for BMP4 RT-PCR analysis is isolated from an additional 5×10⁶ cells. Glycerol cell stocks are prepared for each selected progeny LCL.

LOH Marker Analysis. Genomic DNA is isolated and purified by standard methods, for example using proteinase K, SDS, and phenol. Polymorphic microsatellite repeat sequences are detected by amplification of genomic DNA, preferably by PCR using primer pairs for selected microsatellite markers. For fine mapping, microsatellite markers having average spacing of about 9 cM are used initially, followed by more closely spaced markers to identify markers with LOH as close to the FOP locus as possible.

In a preferred embodiment, one PCR primer of each pair is end-labeled with T4 polynucleotide kinase and y-³²p-ATP. Polymorphic regions are amplified from genomic DNA by PCR using the recommended cycling parameters; conditions are adjusted as necessary to reduce non-specific bands. PCR primers are multiplexed (2-4 pairs per reaction) as possible. The PCR products are denatured at 94° C., resolved on a 6% denaturing polyacrylamide gel and viewed by autoradiography. Genotypes are determined by visual inspection of the band patterns on the autoradiogram.

RNA Isolation. Total RNA is isolated from the cells using isolation techniques and reagents known to the ordinarily skilled artisan following standard protocols. The integrity of the purified RNA is checked by agarose gel electrophoresis on denaturing gels with formaldehyde, and stored at high concentrations (>1 μg/μl) at −70° C.

RT-PCR to detect endogenous BMP4 mRNA levels. Using standard conditions known in the art, first strand cDNA is synthesized from total RNA. Preferably, 1 μg of total RNA is processed using a reverse transcriptase and oligo (dT) primers, at 42° C., 1 hour. Preferably, an RNase inhibitor is included in the reaction, and the RNA is treated with DNAse I prior to the addition of the reverse transcriptase, in order to remove any potential genomic DNA contamination. A portion of the first strand cDNA reaction is used for PCR amplification, preferably by ³²P-labeled gene-specific primers, under standard PCR conditions using Taq polymerase. PCR primers for human BMP4 have been designed and optimized PCR conditions are known. The labeled PCR products are quantitated by radioisotopic analysis, for example using PhosphorImager scanning, and by normalization to a RT-PCR amplified GAPDH mRNA internal standard, and compared to control cell lines. Primers are custom synthesized.

Experimental Alternatives. Applicants anticipate that only one chromosomal region is identified by the inventive method as the FOP mutant gene locus. Homologous recombination involving a gene in a hypothetical pathway between the FOP mutant gene and BMP4 over-expression (FOP gene→A→B→C→ . . . →BMP4 gene) is not predicted to lead to loss of BMP4 over-expression because such a recombination event would exchange a normal allele from one parent with the normal allele from the other parent. A mutation that completely disrupts a gene in the pathway could identify an alternate locus that causes loss of the FOP phenotype, however such an event is expected to be caused by a gene-specific mutation such as a point mutation or deletion, and is expected to be rare and not cause frequent events of LOH on a specific chromosome.

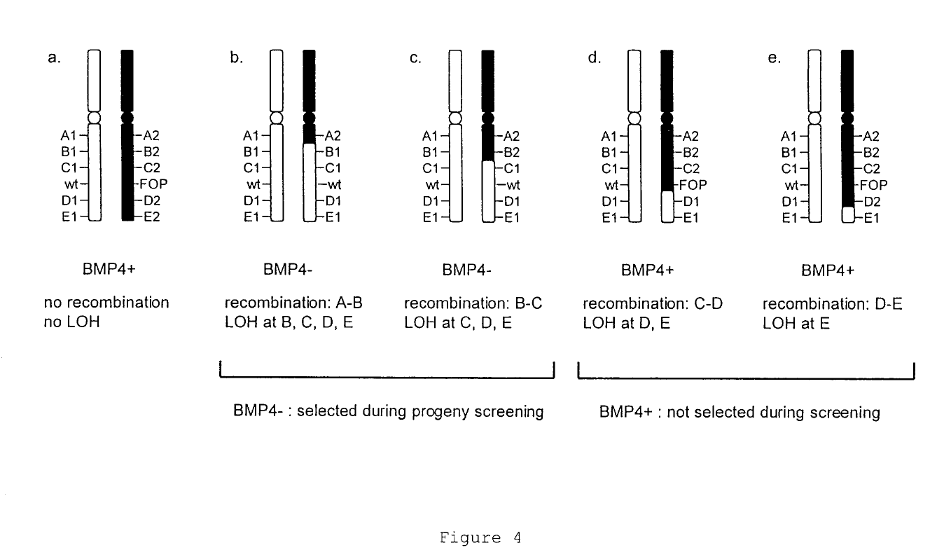

Recombination sites between the centromere and the FOP gene locus are expected to produce cells with LOH of markers distal to the recombination site. A distal (telomeric) marker for each chromosome arm is used in the initial analysis to identify the chromosome arm having marker LOH correlated with the loss of BMP4 expression (see FIG. 4). Markers spaced at ˜10 cM intervals along the length of that arm are expected to more specifically localize the mutant locus. The most distal LOH recombinant with this marker set is fine-mapped to position the marker with LOH as close to the recombination site as possible and to define a proximal boundary for the FOP locus (FIG. 4). The distal boundary is predicted based on the known order of the markers. If the LOH data position the FOP locus on a characterized chromosome, such family linkage data contributes to FOP locus boundary definition. Note that the closer the FOP locus is to a centromere, the lower the chance that a random recombination event occurs between the locus and the centromere; this may require that the numbers of progeny cells that are analyzed be increased. If necessary, site specific recombination is induced to help identify the FOP locus.

The only condition under which Applicants expect that no appropriate recombinant cells are recovered is if LOH of the chromosome region including and distal to the FOP locus results in a lethal phenotype. This could occur through the unmasking of a recessive phenotype on the maternal chromosome or the presence of an imprinted locus; these events are expected to be highly unlikely. Further, preferably, multiple sources of “parent” cells are tested, and not all would be expected to produce a lethal phenotype. Alternate cell line(s) from another source can also be tested if imprinting is suspected. The success of TPA-induced mitotic recombination without identification of cells that have lost BMP4 expression is determined by examination of LOH at another locus, such as HLA-A2.

Applicants have successfully increased the sensitivity and consistency of luciferase reporter gene assays in LCLs through the use of the specific transfection reagents and the reporter systems. In the present invention, it is expected that even a low number of transfected “parent” cells can be identified through antibiotic selection and fluorescent marker expression, and sorted through FACS analysis.

Adenoviral reporter gene constructs are known to be an alternative to plasmid transfection of LCLs. An adenovirus-GFP vector is known. At the optimal viral particles per cell, Applicants expect an adenoviral reporter uptake efficiency of at least 20-25%. When plasmid transfection is not amenable, viral infection is a viable alternative.

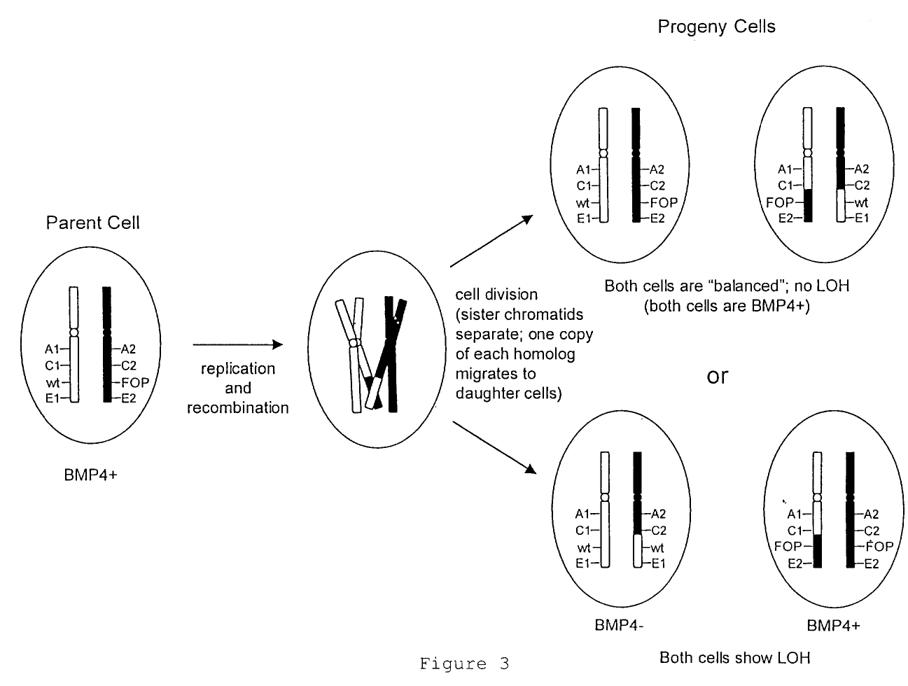

Expected Results. FIG. 3 depicts a “parental” cell from an FOP patient that undergoes homologous recombination during mitosis to produce two progeny cells. FIG. 2 shows how a homologous recombination event that includes the chromosomal region that contains the mutated gene locus in FOP results in an alteration of the cellular “FOP phenotype”. FIG. 3 demonstrates how that FOP gene locus is correlated with a specific chromosome region. A set of highly polymorphic “microsatellite markers” have been identified across the entire human genome. PCR primer pairs are used to amplify a target marker sequence and marker alleles are distinguished through gel electrophoresis.

In FIG. 4, three marker loci (A, C, E) are depicted. The light chromosome contains allele A1, allele C1, and allele E1 at the three loci. The dark chromosome (the homolog of the light chromosome) contains the same three marker loci, but different alleles at each locus (allele A2, allele C2, and allele E2). Each progeny cell is screened by PCR analysis of the markers. When the progeny cell has a “balanced” pair of homologous chromosomes (upper right of figure), the PCR reaction detects both alleles at each locus. However, if the progeny contain chromosome segments that are derived from only one parental homolog (lower right of figure), the PCR products from the markers within that region reflect the presence of only one allele. This is described as loss of heterozygosity. A progeny cell that contains two mutated FOP alleles, assuming that such a cell is viable, retains the BMP4+ FOP phenotype and is not distinguishable from a cell with no recombination or recombination at a site remote from the FOP locus. However, a cell that has two normal alleles would lose the FOP phenotype and this cell is identified and examined to the specific marker loci that show LOH. Since the precise chromosomal location of each marker has been determined, the chromosome region containing the FOP gene is identified.

### Example 4

Transfection of LCLs

A nested set of recombinant chromosomes that correlate with the loss of the mutant phenotype is used to identify the mutant locus. This chromosomal locus must be between the most distal, or telomeric, marker that shows recombination affecting marker expression, defining the proximal boundary, and the most distal marker that does not, defining the distal boundary. The identified chromosome region is examined for candidate mutant genes.

The sites of homologous recombination and loss of the mutant phenotype are correlated with the human genome sequence. Human sequence database(s) are searched for the DNA sequence that corresponds to the identified chromosome region bearing the mutation, i.e. the sequence between the identified border markers. The genes that reside within this interval are identified in silico and candidate genes that could plausibly cause the genetic mutation are screened for mutations, preferably by PCR and DNA sequencing or by other genotyping strategies.

Additional markers in the LOH region are used to “fine map” the site of homologous recombination in order to narrow the interval of interest as much as possible. If the selection of candidate genes within the “linked” interval is very large, polymorphic markers that are tightly linked to individual candidate genes can be used to evaluate linkage of these loci in the mutant cell line(s). When necessary, additional cellular progeny are generated, in the same or different parent cell lines, to reduce the size of the linked interval.

Exemplary Methods in FOP

Database searches. Human genome sequence database(s), for example through the National Center for Biotechnology Information web site, are searched to identify the appropriate DNA sequence and mapped genes. Primary candidate genes are those that are known to influence the BMP signaling pathway.

PCR amplification of genomic DNA. Oligonucleotide primers that flank exon-coding regions of the candidate genes are designed from available DNA sequence databases and used to amplify genomic DNA templates, for example by PCR using optimized amplification conditions. In a preferred embodiment, in a final volume of 50 μl each PCR reaction contains about: 250 ng of genomic DNA, 0.5 μM of each primer, 0.1 mM PCR buffer with optimized MgCl₂, and 1.25 units of Taq polymerase.

DNA sequencing of PCR products. Following amplification, the PCR products are purified. 50-100 ng of DNA is sequenced, preferably using an automated sequencer.

Linkage of candidate genes within FOP families. Genomic DNA is isolated and purified by standard methods from established lymphoblastoid cell lines (LCLs). Polymorphic markers within the chromosome region for the candidate gene of interest will be identified by searching genome sequence databases. Primer pairs are available commercially or are custom synthesized. Genotyping is conducted.

Experimental Alternatives. With sufficient numbers of recombinant progeny, it is expected that an FOP linked region of ˜1-2 cM is identified. If the target region is large, the analysis can be repeated to generate additional recombinants to narrow the target interval. However, with the recent publication of the complete sequence of the human genome, essentially all of the genes within a target interval can be identified, and a rational evaluation of the most likely candidate genes can select at least an initial set of genes to be screened in patients for mutations.

Expected Results. Referring to FIG. 4, the location of the mutant gene between two specific marker loci is examined by considering the order of the marker loci within a nested set of markers showing LOH. In FIG. 4, a pair of homologous chromosomes from each of five progeny cells is shown.

(a) A progeny cell that contains no homologous recombination event has two alleles detected at each of the five loci indicated (A to E).

(b) Recombination caused by a chromosome break between markers A and B results in LOH of markers B, C, D, and E.

(c) Recombination caused by a chromosome break between markers B and C results in LOH of markers C, D, and E.

(d) Recombination caused by a chromosome break between markers C and D results in LOH of markers D and E.

(e) Recombination caused by a chromosome break between markers D and E results in LOH of marker E.

In FIG. 4, cells (d) and (e) have retained the mutant FOP phenotype (BMP4+); therefore it is predicted that the FOP gene locus is not between the marker D locus and the distal telomere. However, cells (b) and (c) have lost the BMP4 FOP phenotype; the FOP locus is predicted to occur between the marker C locus (the most distal marker showing LOH correlated with the loss of the FOP phenotype) and marker D (for which LOH is not associated with loss of the FOP phenotype). Of the cells depicted, only cells (b) and (c) would be selected in the screen for loss of the FOP cellular phenotype.

Because recombination could occur between marker locus C and the FOP locus and not show LOH for marker C, the candidate FOP locus would need to include the region up to the penultimate distal marker showing LOH with loss of the FOP phenotype. Fine mapping with closely spaced markers identifies this proximal border very close to the FOP gene.

All publications and patent documents referred to herein are incorporated by reference in full, to the same extent as if each individual document was specifically and individually indicated to be incorporated by reference.

The invention being thus described, it will be obvious that the same may be modified or varied in many ways. Such modifications and variations are not to be regarded as a departure from the spirit and scope of the invention and all such modifications and variations are intended to be included within the scope of the following claims.

We claim:

1. A method for genetic linkage analysis, comprising:
(a) identifying one or more disease phenotype marker(s), each of which is linked to a disease, disorder, or condition of interest;
(b) inducing mitotic recombination in a parent cell line having a mutated gene to produce recombinant progeny cells;
(c) screening said progeny cells for recombinant cellular DNA by selecting those cells having a loss or gain of one or more of said disease phenotype marker(s); and
(d) identifying chromosomal site(s) of recombination within said progeny cells having a loss or gain of disease phenotype marker(s).

2. The method of claim 1, wherein said method further is for identification of a mutated gene, comprising the additional steps of:
(e) identifying one or more candidate gene(s) for said mutated gene within said chromosomal site(s) of recombination; and
(f) screening each said candidate gene for mutations.

3. The method of claim 1, wherein said mitotic recombination in a parent cell line is induced by treatment of a parent cell with 12-0-tetradecanolyphorbol-13-acetate, ethylmethanesulfonate, UV irradiation, X-ray irradiation, or a combination thereof.

4. The method of claim 1, wherein said mitotic recombination in a parent cell line is induced by treatment of a parent cell with 12-0-tetradecanolyphorbol-13-acetate.

5. The method of claim 1, wherein said loss of one or more disease phenotype marker(s) is produced by a loss of heterozygosity within said progeny cells.

6. The method of claim 1, wherein said chromosomal site(s) of recombination within said progeny cells having a loss of disease phenotype marker(s) are identified using chromosomal microsatellite markers.

7. The method of claim 1, wherein each said disease phenotype marker is selected from the group consisting of protein over-expression, protein under-expression, expression of a defective protein, lack of protein expression, over-production of mRNA, or under-production of mRNA.

8. The method of claim 2, wherein said candidate gene(s) are identified by positional cloning or database searching.

9. The method of claim 2, wherein said screening is achieved by a selection process.

10. A method for genetic linkage analysis, comprising:
(a) identifying one or more disease phenotype marker(s), each of which is linked to a disease, disorder, or condition of interest;
(b) inducing mitotic recombination in a parent cell line having a mutated gene to produce recombinant progeny cells,
wherein said mitotic recombination is induced by induced by treatment of a parent cell with 12-0-tetradecanolyphorbol-13-acetate;
(c) screening said progeny cells for recombinant cellular DNA by selecting those cells having a loss or gain of one or more of said disease phenotype marker(s),
wherein said loss of one or more disease phenotype marker(s) is produced by a loss of heterozygosity within said progeny cells; and
(d) identifying chromosomal site(s) of recombination within said progeny cells having a loss or gain of disease phenotype marker(s),
wherein said chromosomal site(s) of recombination within said progeny cells having a loss of disease phenotype marker(s) are identified using chromosomal microsatellite markers.

11. The method of claim 10, wherein said method further is for identification of a mutated gene, comprising the additional steps of:
(e) identifying one or more candidate gene(s) for said mutated gene within said chromosomal site(s) of recombination within said progeny cells,
wherein each said candidate gene is identified by positional cloning or database searching; and
(f) screening each said candidate gene for mutations.

12. The method of claim 10, wherein each said disease phenotype marker is selected from the group consisting of protein over-expression, protein under-expression, expression of a defective protein, lack of protein expression, over-production of mRNA, or under-production of mRNA.

13. A method for genetic linkage analysis and identification of a mutated gene linked with Fibrodysplasia Ossificans Progressiva, comprising:
(a) inducing mitotic recombination in a parent cell line having a mutated gene linked with Fibrodysplasia Ossificans Progressiva to produce recombinant progeny cells,
wherein said mitotic recombination is induced by induced by treatment of a parent cell with 12-0-tetradecanolyphorbol-13-acetate;
(b) screening said progeny cells for recombinant cellular DNA by selecting those cells having a loss of BMP4 over-expression, loss of BMP antagonist response to a BMP4 stimulus, or decrease in the density of BMP receptor BMPRIA,
wherein said loss of BMP4 over-expression, loss of BMP antagonist response to a BMP4 stimulus, or decrease in the density of BMP receptor BMPRIA is produced by a loss of heterozygosity within said progeny cells;
(c) identifying chromosomal site(s) of recombination within said progeny cells having a loss of BMP4 over-expression, loss of BMP antagonist response to a BMP4 stimulus, or decrease in the density of BMP receptor BMPRIA,
wherein said chromosomal site(s) of recombination within said progeny cells having a loss of BMP4 over-expression, loss of BMP antagonist response to a BMP4 stimulus, or decrease in the density of BMP receptor BMPRIA are identified using chromosomal microsatellite markers;
(d) identifying one or more candidate gene(s) for said mutated gene linked with Fibrodysplasia Ossificans Progressiva within said chromosomal site(s) of recombination within said progeny cells,
wherein each said candidate gene is identified by positional cloning or database searching; and
(e) screening each said candidate gene for mutations.

14. A kit for producing a clonally-expanded, nested cellular progeny cell set from a parent cell, comprising:
(a) a means for obtaining and isolating a parent cell line from an individual;
(b) a means for inducing mitotic recombination in said parent cell line to produce progeny cells;
(c) a means for growing said progeny cells; and
(d) a means for screening said progeny cells for recombinant progeny cells.

15. The kit of claim 14, wherein said means for inducing mitotic recombination is selected from the group consisting of 12-0-tetradecanolyphorbol-13-acetate, ethylmethanesulfonate, UV irradiation, X-ray irradiation, and a combination thereof.

16. The kit of claim 14, wherein said means for inducing mitotic recombination comprises 12-0-tetradecanolyphorbol-13-acetate.

17. An apparatus for genetic linkage analysis of a cell of an organism having a genetic mutation of interest and a linked disease phenotype marker, comprising:
(a) a first stage comprising a system for inducing mitotic recombination in a parent cell line to produce clonally-expanded progeny cells;
(b) a second stage comprising a cell sorting system for selection for the loss or gain of expression of said disease phenotype marker;
(c) a third stage comprising an antibiotic selection system for selection of cells that contain said disease phenotype marker;
(d) a fourth stage comprising an isolation system for isolating genomic DNA from said progeny cells;
(e) a fifth stage comprising a screening system for screening said genomic DNA for one or more genomic marker(s); and
(f) a sixth stage comprising a system for correlating said sites of homologous recombination and loss or gain of said mutant phenotype with the genomic DNA sequence of said organism.

18. The apparatus of claim 17, wherein said means for inducing mitotic recombination is selected from the group consisting of 12-0-tetradecanolyphorbol-13-acetate, ethylmethanesulfonate, UV irradiation, X-ray irradiation, and a combination thereof.

19. The apparatus of claim 17, wherein said means for inducing mitotic recombination comprises 12-0-tetradecanolyphorbol-13-acetate.

20. A DNA sequence containing a mutation, identified by a process comprising:
(a) inducing mitotic recombination in a parent cell line having a mutated gene linked with Fibrodysplasia Ossificans Progressiva to produce recombinant progeny cells,
wherein said mitotic recombination is induced by induced by treatment of a parent cell with 12-0-tetradecanolyphorbol-13-acetate;
(b) screening said progeny cells for recombinant cellular DNA by selecting those cells having a loss of BMP4 over-expression, loss of BMP antagonist response to a BMP4 stimulus, or decrease in the density of BMP receptor BMPRIA, wherein said loss of BMP4 over-expression, loss of BMP antagonist response to a BMP4 stimulus, or decrease in the density of BMP receptor BMPRIA is produced by a loss of heterozygosity within said progeny cells;
(c) identifying chromosomal site(s) of recombination within said progeny cells having a loss of BMP4 over-expression, loss of BMP antagonist response to a BMP4 stimulus, or decrease in the density of BMP receptor BMPRIA, wherein said chromosomal site(s) of recombination within said progeny cells having a loss of BMP4 over-expression, loss of BMP antagonist response to a BMP4 stimulus, or decrease in the density of BMP receptor BMPRIA are identified using chromosomal microsatellite markers;
(d) identifying one or more candidate gene(s) for said mutated gene linked with Fibrodysplasia Ossificans Progressiva within said chromosomal site(s) of recombination within said progeny cells,
wherein each said candidate gene is identified by positional cloning or database searching;
(e) sequencing each said candidate gene;
(f) comparing the DNA sequence for each candidate gene with a wild-type DNA sequence of said gene; and
(g) identifying said DNA sequence containing a mutation for each said candidate gene.

21. A method for treating Fibrodysplasia Ossificans Progressiva, comprising:
(a) identifying a defective gene responsible for causing Fibrodysplasia Ossificans Progressiva;
(b) harvesting somatic parent cells from an individual having a mutated gene producing the phenotype for Fibrodysplasia Ossificans Progressiva;
(c) producing recombinant progeny cells by a method of inducing mitotic recombination in said parent cells line having a mutated gene;
(d) screening said recombinant progeny cells for recombinant cells having a wild-type copy of said defective gene; and
(e) re-introducing said recombinant cells having a wild-type copy of said defective gene into said individual.

22. The method of claim 21, wherein said defective gene responsible for causing Fibrodysplasia Ossificans Progressiva is identified by the method of claim 2.

23. A method for treating Fibrodysplasia Ossificans Progressiva in an individual in need of thereof, comprising:
(a) identifying a defective gene responsible for causing Fibrodysplasia Ossificans Progressiva; and
(b) administering a composition which upregulates the expression of a wild-type gene, down regulates the expression of said defective gene, or indirectly sequesters or deactivates a defective protein produced by expression of said defective gene.

24. The method of claim 23, wherein said defective gene responsible for causing Fibrodysplasia Ossificans Progressiva is identified by the method of claim 2.

25. The method of claim 23, wherein said composition for upregulation of the expression of a wild-type gene is a vector encoding, or a delivery system carrying, one or more copies of an isolated exogenous wild-type copy of said defective gene which is capable of being expressed in said individual.

26. The method of claim 25, wherein said vector or delivery system is selected from the group consisting of an adenovirus vector, a lentivirus vector, a lentiviral/adenoviral chimeric vector, and a liposome packaging and delivery system.

27. The method of claim 26, wherein said vector is a replication-defective adenovirus vector.
